(19) United States
(12) Patent Application Publication
(10) Pub. No.: US 2026/0078348 A1
(43) Pub. Date: Mar. 19, 2026

(54) MULTIPLE MYELOMA MICROORGANOSPHERES

(71) Applicant: Xilis, Inc., Durham, NC (US)

(72) Inventors: Xiling Shen, Chapel Hill, NC (US); Shengli Ding, Durham, NC (US)

(21) Appl. No.: 19/108,545

(22) PCT Filed: Sep. 7, 2023
(86) PCT No.: PCT/US2023/032182

§ 371 Date: Mar. 4, 2025

Related U.S. Application Data
(60) Provisional application No. 63/404,472, filed on Sep. 7, 2022.

(51) Int. Cl. C12N 5/09; G01N 33/50

(52) U.S. Cl. CPC C12N 5/0694; G01N 33/5011; C12N 2503/02; C12N 2513/00

(57) ABSTRACT

MicroOrganoSpheres (MOS) generated using cells from multiple myeloma bone marrow biopsies are provided herein, as are methods and materials for making and using such MOS.

### CROSS-REFERENCE TO RELATED APPLICATIONS

This application claims benefit of priority from U.S. Provisional Application Ser. No. 63/404,472, filed Sep. 7, 2022. The disclosure of the prior application is considered part of (and is incorporated by reference in) the disclosure of this application.

### TECHNICAL FIELD

This document relates to MicroOrganoSpheres (MOS) generated from bone marrow biopsies that contain multiple myeloma (MM) cells, and to methods and materials for making and using MM-containing MOS.

### BACKGROUND

MM is a plasma cell malignancy with an estimated incidence of five to seven new cases per 100,000 people, making it the second most common hematological malignancy in the United States. In 2019 alone, there were about 32,000 new cases of MM and 12,960 deaths from MM in the United States. Despite improvements in the treatment and overall outcomes of patients with MM over the last several decades, MM remains an incurable disease, and nearly all patients will continuously undergo cycles of treatment, response, and relapse. For example, almost all patients with MM require life-long treatment including a two to four drug combination therapy as well as single agent maintenance therapy. FDA-approved drugs for treatment of MM include immunomodulatory agents (e.g., IMiDs, thalidomide, lenalidomide, and pomalidomide), proteasome inhibitors (PIS) (e.g., bortezomib, carfilzomib, and ixazomib), monoclonal antibodies (e.g., elotuzumab, daratuzumab, isatuximab, and belantamab), nuclear exporter inhibitors (e.g., selinexor), doxorubicin, panobinostat, and melflufen. Steroids (e.g., dexamethasone) and alkylating agents (e.g., cyclophosphamide, melphalan, and bendamustine) also are commonly used for treating MM. In addition, chimeric antigen receptor (CAR) T cell therapies (e.g., Abecma, Ide-cel) have been approved by the U.S. Food and Drug Administration, and bi-specific antibodies may soon be used as a standard treatment for MM.

### SUMMARY

This document is based, at least in part, on the development of methods for generating, testing, and validating Patient-Derived MicroOrganoSpheres (PDMOs) generated from MM biopsies (also referred to herein as MM MOS). This document also is based, at least in part, on the use of MM MOS with a platform referred to as MicroOrganoSphere Drug Screen to Lead Care (MODEL), which can serve as a diagnostic assay to identify appropriate treatments for, for example, relapsed/refractory (RR) MM patients and naïve MM patients, such that therapeutic regimens can be matched with individual patients. The methods and materials provided herein can facilitate rapid diagnosis (e.g., within two weeks or less), are cost effective and readily used, and can provide for high throughput screening of treatments for individual MM patients. The availability of numerous therapeutic agents for treating MM (e.g., the agents listed above) has made it challenging to select, combine, and sequentially use these agents. Having the ability to select the appropriate agent(s) and the appropriate drug combination(s) for a particular patient can facilitate successful management of patients with MM.

This document provides methods and materials for generating MOS from bone marrow biopsies obtained from mammals (e.g., humans) with MM. For example, this document provides methods that include obtaining a bone marrow biopsy from an MM patient, using the obtained bone marrow tissue to prepare a population of MOS, and screening the MOS for response to various MM treatments, thus identifying which of the tested therapies might be most effectively used in the patient.

As demonstrated herein, MOS were successfully generated from fresh bone marrow biopsies from MM patients. The MOS were contacted with several therapeutic agents to determine the effect of those drugs on the MOS, revealing a differential effect on the MOS depending on which drug was used. In addition, further studies demonstrated that the MM MOS could withstand freezing and thawing.

In a first aspect, this document features a MicroOrganoSphere containing bone marrow cells from a mammal with multiple myeloma (MM). The MicroOrganoSphere can contain about 50 to about 150 cells, about 75 to about 125 cells, or about 100 cells. The cells can include cancer cells, stromal cells, stem cells, immune cells, or any combination thereof. The MicroOrganoSphere can contain cancer cells and stromal cells at a ratio of less than about 1:4. The immune cells can include at least one macrophage. The MicroOrganoSphere can contain a solubilized basement membrane matrix.

In another aspect, this document features a composition containing a MicroOrganoSphere in a culture medium, where the MicroOrganoSphere contains bone marrow cells from a mammal with MM. The culture medium can include a solubilized basement membrane matrix. The composition can contain about 1% of the solubilized basement membrane matrix. The composition can further contain an immiscible fluid (e.g., an oil).

In another aspect, this document features a method for making MicroOrganoSpheres (MOS). The method can include (a) receiving a bone marrow sample from a mammal having MM; (b) refining the bone marrow sample to form a refined sample; and (c) forming a population of MOS from the refined sample by (i) driving an unpolymerized fluid mixture through one or more channels of a microfluidics apparatus, wherein the unpolymerized fluid mixture includes the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow, (ii) forming a plurality of droplets containing the unpolymerized fluid mixture within the microfluidics apparatus, and (iii) polymerizing the fluid matrix material to form the MOS, wherein the MOS each have a diameter of between 50 and 500 μm with between 30 and 150 cells distributed therein.

The method can further include driving an immiscible fluid through another channel of the microfluidics apparatus, such that the immiscible fluid is combined with the unpolymerized fluid mixture prior to forming the plurality of droplets, wherein the droplets include the unpolymerized fluid mixture and the immiscible fluid. The immiscible fluid can be an oil. The forming the population of MOS can further include sorting the MOS based on cell number and/or droplet size. The sorting can include optical sorting based on cell number and/or droplet size. The forming the population of MOS can include forming about 100 to about 600 MOS, forming about 600 to about 1,000 MOS, or forming more than about 1,000 MOS. The microfluidics apparatus can maintain a viscosity of the unpolymerized fluid mixture prior to forming the plurality of droplets. The microfluidics apparatus can be configured to prevent clogging of the unpolymerized fluid mixture within the one or more channels. The microfluidics apparatus can be configured to prevent clogging by having channel diameters of 100 μm or greater. The microfluidics apparatus can be configured to maintain an approximately constant pressure within the one or more channels. The microfluidics apparatus can maintain a constant flow rate within the one or more channels. The total length of a path taken by the unpolymerized fluid mixture before the forming of the plurality of droplets within the microfluidics apparatus can be less than 10 cm. The MOS of the population of MOS can have less than a 25% variation in size. The polymerizing can include crosslinking the fluid matrix material. The fluid matrix material can be chemically crosslinkable or photo-crosslinkable. The bone marrow sample can include freshly biopsied cells. The bone marrow sample can have been taken from the mammal within 24 hours of forming the MOS. The bone marrow sample can include MM plasma cells, immune cells, stem cells, stromal cells, or any combination thereof. The immune cells can include one or more of T cells, B cells, macrophages, dendritic cells, NK cells, monocytic cells, and combinations thereof. The method can include flowing the unpolymerized fluid mixture through the one or more channels at a flow rate of about 0.01 milliliter (mL) per minute (min) to about 100 mL/min.

In another aspect, this document features a method of precision drug screening for personalized cancer therapy for MM. The method can include (a) receiving a bone marrow sample from a mammal having MM; (b) refining the sample to form a refined sample; (c) forming a population of MOS from the refined sample by (i) driving an unpolymerized fluid mixture through one or more channels of a microfluidics apparatus, wherein the unpolymerized fluid mixture includes the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow, (ii) forming a plurality of droplets containing the unpolymerized fluid mixture within the microfluidics apparatus, and (iii) polymerizing the fluid matrix material to form the MOS, wherein the MOS each have a diameter of between 50 and 500 μm with between 1 and 500 cells distributed therein; (d) culturing the population of MOS for between 1-14 days; and (e) assaying one or more drug therapies using the population of MOS.

The method can further include driving an immiscible fluid through another channel of the microfluidics apparatus, such that the immiscible fluid is combined with the unpolymerized fluid mixture prior to forming the plurality of droplets, wherein the droplets include the unpolymerized fluid mixture and the immiscible fluid. The immiscible fluid can be an oil. The assaying can include assaying, in parallel, a plurality of drug therapies by exposing one or more of the MOS to each drug therapy. The method can include characterizing a response of the MOS to each of the plurality of drug therapies based on a response of the MOS to exposure to the plurality of drug therapies. The time between receiving the bone marrow sample and characterizing the response can be less than 21 days. The forming the population of MOS can further include sorting the MOS based on cell number and/or droplet size. The sorting can include optically sorting the MOS or based on cell number and/or droplet size. The assaying can include assaying more than 10 different drug therapies. The one or more drug therapies can include different concentrations of one or more drug, different combinations of two or more drugs, different ratios of two or more drugs, different carriers for one or more drug, and/or different dose times for one or more drugs. The forming the population of MOS can include forming about 100 to about 600 MOS, forming about 600 to about 1,000 MOS, or forming more than 1,000 MOS. The microfluidics apparatus can maintain a viscosity of the unpolymerized fluid mixture prior to forming the plurality of droplets. The microfluidics apparatus can be configured to prevent clogging of the unpolymerized fluid mixture within the one or more channels. The microfluidics apparatus can be configured to prevent clogging by having channel diameters of 100 μm or greater. The microfluidics apparatus can be configured maintain an approximately constant pressure within the one or more channels. The microfluidics apparatus can maintain a constant flow rate within the one or more channels. The total length of a path taken by the unpolymerized fluid mixture before the forming of the plurality of droplets within the microfluidics apparatus can be less than 10 cm. The method can further include measuring an effect of the one or more drug therapies on cells within the MOS. The method can further include determining that the mammal is still responding to a drug therapy of the one or more drug therapies after one or more administrations of the drug therapy, by receiving a second bone marrow sample after the mammal has been treated with the drug therapy and forming a second population of MOS from the second bone marrow sample, exposing at least some of the second population of MOS to the drug therapy, and measuring an effect of the drug therapy on cells within the at least some of the second population of MOS. The method can further include treating the mammal with a drug therapy of the one or more drug therapies. The MOS of the population of MOS can have less than a 25% variation in size. The polymerizing can include crosslinking the fluid matrix material. The fluid matrix material can be chemically crosslinkable or photo-crosslinkable. The bone marrow sample can include freshly biopsied cells. The bone marrow sample can have been taken from the mammal within 24 hours of forming the MOS. The bone marrow sample can include cancer cells, immune cells, stem cells, stromal cells, or any combination thereof. The immune cells can include one or more of T cells, B cells, macrophages, dendritic cells, NK cells, monocytic cells, and combinations thereof. The method can include flowing the refined bone marrow sample and the unpolymerized fluid matrix through the one or more channels at a flow rate of about 0.01 mL/min to about 100 mL/min.

Unless otherwise defined, all technical and scientific terms used herein have the same meaning as commonly understood by one of ordinary skill in the art to which this invention pertains. Although methods, devices, and materials similar or equivalent to those described herein can be used to practice the invention, suitable methods, devices, and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C are representative images showing PDMOs formed to include a single primary tissue cell per MOS, cultured for one day after forming (FIG. 1A), three days after forming (FIG. 1B), and seven days after forming (FIG. 1C). The cells originated from colorectal cancer (CRC) tissue.

FIGS. 2A-2C are representative images showing PDMOs formed to include five primary tissue cells per MOS, cultured for one day after forming (FIG. 2A), three days after forming (FIG. 2B), and seven days after forming (FIG. 2C). The cells originated from CRC tissue.

FIGS. 3A-3C are representative images showing PDMOs formed to include twenty primary tissue cells per MOS, cultured for one day after forming (FIG. 3A), three days after forming (FIG. 3B), and seven days after forming (FIG. 3C). The cells originated from CRC tissue.

FIGS. 4A to 4E include images showing representative examples of PDMOs formed to include ten primary tissue cells per MOS. FIG. 4A shows the MOS shortly after formation (at low magnification). FIG. 4B shows a higher magnification view of some of the MOS of FIG. 4A, taken after culturing for two days. FIG. 4C shows the MOS after culturing for three days. FIG. 4D shows the MOS after culturing for four days. FIG. 4E shows the MOS after culturing for five days.

FIGS. 5A to 5B are images of representative examples of MOS formed from normal mouse liver hepatocytes, cultured for one day after forming (FIG. 5A) or for ten days after forming (FIG. 5B), The mouse hepatocytes were taken from a normal (non-diseased) mouse liver.

FIG. 6 is a schematic illustrating the steps of a method for forming PDMOs from primary tissue (e.g., biopsy) samples.

FIG. 7A is a schematic illustrating an example of an apparatus for forming PDMOs, including a microfluidic chip as part of the assembly. FIG. 7B is a perspective view of one example a microfluidics chip portion of an apparatus such as that shown in FIG. 7A. FIG. 7C is a schematic illustrating a portion of a microfluidics assembly for an apparatus for forming PDMOs, such as the one shown in FIG. 7A.

FIG. 8 is an image showing a plurality of PDMOs formed using an apparatus such as that shown in FIG. 7A, showing the PDMO shortly after polymerizing, suspended within a channel containing the immiscible fluid (e.g., oil) prior to being aliquoted from the apparatus.

FIG. 9 is an image of a portion of a prototype microfluidics assembly for an apparatus for forming PDMOs, similar to that shown in FIG. 7C, illustrating the formation of PDMOs.

FIG. 10 is an image showing a plurality of PDMOs shortly after polymerization; the PDMOs are suspended in the immiscible fluid.

FIGS. 11A and 11B are images showing a plurality of PDMOs shortly after formation and suspension in an immiscible fluid (e.g., oil) at low magnification (FIG. 11A) and higher magnification (FIG. 11B).

FIGS. 12A and 12B are images showing a plurality of PDMOs following separation from the immiscible fluid within a few hours of formation of the PDMOs at low magnification (FIG. 12A) and higher magnification (FIG. 12B).

FIG. 13 is an image showing another example of a plurality of PDMOs.

FIG. 14 is a graph plotting the size distribution of the diameters of a plurality of PDMOs formed from an exemplary biopsy sample.

FIGS. 15A and 15B are images showing low and higher magnification views, respectively, of an example of a plurality of PDMOs formed from a dissociated tissue biopsy sample and a fluid matrix material, after polymerizing. FIG. 15A is an unstained image, while in FIG. 15B the MOS were stained with Trypan blue to show that the cells in the MOS are alive.

FIGS. 16A and 16B are images showing another example of low and higher magnification views, respectively, of an example of a plurality of PDMOs. FIG. 16A is an unstained image, while in FIG. 16B the MOS were stained with Trypan blue (arrows) to show that the cells in the MOS remained viable (e.g., alive) within the MOS.

FIGS. 17A-17E illustrate an example of a method for assaying a plurality of PDMOs formed from a patient tumor biopsy, to determine a drug-response profile to multiple drug formulations. The illustrated procedure takes less than two weeks from biopsy to results.

FIG. 18 is a schematic illustrating an example of a method for treating a patient, including the formation and use of a plurality of PDMOs as part of the treatment procedure.

FIG. 19 is a schematic illustrating an example of a method for treating a patient, including multiple iterations of rapidly forming and assaying a plurality of PDMOs as part of the treatment procedure.

FIG. 20A includes representative images showing the formation of MM MOS from human bone marrow over the course of 11 days. MM MOS are indicated by the white arrows. FIG. 20B includes representative images showing that without stromal cells, CD138+ cells (isolated from bone marrow cells using bead selection) did not form MOS.

FIGS. 21A-21E show that established MM MOS preserved major cell populations from bone marrow. FIG. 21A is a flow cytometry plot for single cells from a bone marrow biopsy sample and in MOS cultured in vitro for 9 days. The myeloma cell population was demonstrated to be CD11b− and CD38+. FIGS. 21B and 21C are a pair of flow cytometry plots showing that the major immune cell populations were preserved in day 8 MOS (FIG. 21C) as compared to bone marrow biopsy (FIG. 21B). Flow cytometry was performed for day 3 and day 8 MOS, and the overall cell percentages over time during MOS culture are plotted in the graph shown in FIG. 21D. FIG. 21E includes additional flow cytometry plots showing that established MM MOS preserved major cell populations from bone marrow. Flow cytometry was performed on day 11 MM MOS. Plasma cells and MM cells (CD138⁺ and CD38⁺, respectively), T cells (CD3⁺, CD4⁺, and CD8⁺, respectively), and dendritic cells (CD11b⁺) were identified, as indicated.

FIG. 22A includes representative images showing MOS containing 50, 70, or 100 cells, at days 0 and 7. FIG. 22B includes representative images of day 7 MOS stained for live cells.

FIG. 23A is a graph plotting the Caspase 3/7 signal in MM MOS after treatment with lenalidomide, bortezomib, or negative control for 92 hours. MM MOS death is indicated by the increased Caspase 3/7 signal. FIG. 23B includes representative INCUCYTE® images showing the MM MOS response to lenalidomide as early as 24 hours after drug treatment.

FIGS. 24A and 24B show that MM MOS responded to carfilzomib and selinexor treatment. FIG. 24A includes representative live/dead dye images of MOS after treatment with 10 μM carfilzomib or 10 μM selinexor for 4 days. Calcein acetoxymethyl ester (calcein AM) (green) was used to stain live cells, and ethidium homodimer (red) was used to stain dead cells. FIG. 24B is a graph plotting the live/dead ratios. n=3 for each treatment group. *p<0.05 by t-test.

FIG. 25A includes a pair of images showing MM MOS after a freeze thaw cycle, indicating that the MOS remained viable. FIG. 25B includes plots showing viability of the freeze-thawed MM MOS as demonstrated by SYTOX™ blue-measurement via flow cytometry.

### DETAILED DESCRIPTION

Model cell and tissue systems, including three-dimensional (3D) aggregates such as spheroids and organoids, can be useful for biological and medical research. For example, 3D cell culture models have been helpful in developmental biology, disease pathology, regenerative medicine, drug toxicity and efficacy testing, and personalized medicine. Patient-derived models of cancer (PDMC), such as cell lines, organoids and patient-derived xenografts (PDXs) can provide preclinical models to facilitate the identification and development of new therapeutics, and to predict drug response and identify novel drug combinations. For example, large-scale drug screens of cell lines and organoids derived from cancer patients can be used to identify sensitivity to a large number of potential therapeutics.

Multicellular tumor spheroids can be obtained by culturing cancer cell lines under non-adherent conditions. Spheroids typically form from cancer cell lines as freely floating cell aggregates in ultra-low attachment plates. Spheroids have been shown to maintain more stem cell associated properties than two-dimensional (2D) cell cultures. Organoids are in vitro derived aggregates that include a population of stem cells that can differentiate into cells of major cell lineages. Organoids typically have a diameter greater than one millimeter, and can be cultured through passages. Organoids typically are slower to grow and expand than 2D cell cultures. The generation of organoids from clinical samples requires a sufficient initial number of viable cells (generally hundreds to thousands of cells), and it therefor can be challenging to derive organoids from low volume samples such as biopsies.

Although precision medicine strategies have been developed through the exploration of these various PDMC models, there are barriers to their effective use. Patient-derived organoids (PDO) are believed to be the most accurate in depicting patient tumors, as studies have shown that phenotypic and genotypic profiling of organoids often show a high degree of similarity to the original patient tumors, but there are limitations to the use of PDO to guide therapy. For example, it typically takes several months to develop and test drug sensitivity in organoids, which decreases the clinical applicability because ideally, an assay should be performed from a single core biopsy within 7-10 days. In addition, the number of organoids obtained from a clinically relevant 18-gauge core biopsy is generally not sufficient to perform high throughput drug screen.

Moreover, it is not easy to use MM tissue to replicate tumor cells. MM cells typically are difficult to work with, and attempts to culture MM cells in 3D culture are rarely made, particularly since there is no universal recipe for multiple myeloma 3D culture. An additional challenge is presented by the fact that without bone marrow stromal cells, MM cells can't survive. As described herein, however, PDMOs were successfully generated from MM biopsies, which was surprising given the above-referenced challenges. However, the technology described herein can provide a tumor microenvironment for MM cells, thereby allowing the MM cells to grow. Cells within the MM immune microenvironment (e.g., macrophages) also are captured in the MOS made by the methods provided herein, expanding the range of drugs against which the MOS can be screened.

In general, therefore, this document provides MOS generated from bone marrow samples from mammals having MM. In addition, this document provides methods, materials, and apparatuses for forming MM PDMOs (also referred to herein as “MM MOS”), as well as methods, materials, and apparatuses for using the MM PDMOs (e.g., to assay for MM responses to one or more therapeutic agents). The MM MOS and methods of making and using them described herein can address clinical limitations such as those mentioned above.

The MM PDMOs described herein typically are spheres formed from primary cells distributed within the base bone marrow material, after refining of the primary cells to remove non-mononuclear cells (e.g., red blood cells, granulocytes, and/or platelets) while retaining mononuclear cells, thus forming a refined tissue sample that contains myeloma cancer cells in combination with stromal cells, stem cells, immune cells, or any combination thereof. These PDMOs (also referred to as “MM MOS”) can have a diameter of about 50 μm and about 500 μm (e.g., from about 50 μm to about 400 μm, from about 50 μm to about 300 μm, from about 50 μm to about 250 μm, from about 100 μm to about 500 μm, from about 100 μm to about 250 μm, or from about 50 μm to about 200 μm). The MM MOS can initially contain from about 1 to about 1000 primary cells distributed within the base material (e.g., from about 1 to about 750, from about 1 to about 500, from about 1 to about 400, from about 1 to about 300, from about 1 to about 200, from about 1 to about 150, from about 1 to about 100, from about 25 to about 200, or from about 50 to about 150 primary cells).

Surprisingly, despite their small size (often from about 50 to about 250 μm) and low cell density (e.g., often from about 50 to about 200 cells per MM MOS), the MM MOS provide herein can be used immediately or cultured for a relatively short period of time (e.g., 14 days or less, 10 days or less, 7 days or less, or 5 days or less), and can allow the cells within the MM MOS to survive while maintaining most or even all of the characteristics of the tissue from which they were extracted. The survival rate of the cells within the MM MOS typically is high, and the MM MOS can be cultured for days or even weeks. Also surprisingly, in some variations, the cells from the refined bone marrow sample within the MM MOS can form morphological structures inside even the smallest MOS; although in some applications, the presence of such structures is not necessary for the utility of these MOS (such as when they are used before substantial structural reorganization has occurred), in some variations such structures can be particularly useful.

In some cases, the methods and materials described herein for forming and using MM MOS can be used to generate many (e.g., more than 10,000) PDMOs from a single biopsy. The MOS can be used to, for example, screen various therapeutic agents to predict which one(s) might be effectively and safely used in the MM patient from whom the bone marrow biopsy was taken. For example, the MM MOS can be used in toxicity screens for drugs or other chemical compositions, and to determine whether one or more drug compositions might effectively treat a MM patient before the patient undergoes drug therapy. This can allow for very rapid screening of a MM patient before they would otherwise undergo months of chemotherapy that may not be effective.

Thus, provided herein are high-throughput drug screening methods and apparatuses for performing these methods using a single patient-specific MM biopsy. Described herein are droplet formed PDMOs that can be formed from MM patient-derived bone marrow samples that have been refined and suspended in a basement matrix (e.g., MATRIGEL®, a solubilized basement membrane matrix secreted by Engelbreth-Holm-Swarm mouse sarcoma cells, available from Sigma). The MM MOS can be patterned onto a microfluidic microwell array to be incubated and dosed with drug compounds. This miniaturized assay can maximize the use of tumor samples, and can enable more drug compounds to be screened from a bone marrow aspirate at much lower cost per sample.

While the terms used herein are believed to be well understood by those of ordinary skill in the art, definitions are set forth herein to facilitate explanation of the presently disclosed subject matter.

The term “an unpolymerized mixture” is used herein to refer to a composition containing biologically relevant materials, including a refined tissue sample and a first fluid matrix material. The fluid matrix material typically is a material that can be polymerized to form a support or support network for the refined tissue sample and/or cells dispersed within the refined sample. Once polymerized, the polymerized material may form a hydrogel and may be formed and/or may include proteins forming the biocompatible medium, in addition to the cells. A biocompatible medium suitable for use in the methods disclosed herein can be formed from any biocompatible material that is a gel, a semi-solid, or a liquid, such as a low-viscosity liquid, at room temperature (e.g., 25° C.), and can be used as a three-dimensional substrate for cells, tissues, proteins, and other biological materials of interest. Exemplary materials that can be used to form a biocompatible medium in accordance with the presently-disclosed subject matter include, but are not limited to, polymers and hydrogels containing collagen, fibrin, chitosan, MATRIGEL® (BD Biosciences, San Jose, CA), polyethylene glycol (PEG), dextrans (e.g., chemically crosslinkable or photo-crosslinkable dextrans), and the like, as well as electrospun biological, synthetic, or biological-synthetic blends. In some cases, the biocompatible medium can be a hydrogel.

The term “hydrogel” is used herein to refer to two- or multi-component gels having a three-dimensional network of polymer chains, where water acts as the dispersion medium and fills the space between the polymer chains. Hydrogels used in accordance with the presently-disclosed subject matter generally can be chosen for a particular application based on the intended use of the structure, taking into account the parameters that are to be used to form the MM MOS, as well as the effect the selected hydrogel will have on the behavior and activity of the biological materials (e.g., cells) incorporated into the biological suspensions that are to be placed in the structure. Exemplary hydrogels for use with the presently-disclosed subject matter include polymeric materials such as, without limitation, alginate, collagen (including collagen types I and VI), elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxy apatite, calcium phosphate, bone, and any combination thereof.

With further regard to the hydrogels used to produce the MM MOS described herein, in some cases, the hydrogel can contain a material selected from the group consisting of agarose, alginate, collagen type I, a polyoxyethylene-polyoxypropylene block copolymer (e.g., PLURONIC® F127; BASF Corporation, Mount Olive, NJ), silicone, polysaccharide, polyethylene glycol, and polyurethane. In some cases, the hydrogel can be made up of alginate.

The MM MOS provided herein also can contain biologically relevant materials. The phrase “biologically-relevant materials” refers to materials that are capable of being included in a biocompatible medium as defined herein and subsequently interacting with and/or influencing biological systems. For example, in some cases, biologically-relevant materials can be magnetic beads (such as beads that are magnetic themselves or that contain a material that responds to a magnetic field, such as iron particles) that can be combined as part of the unpolymerized material to aid in the production of MM MOS (e.g., for the separation and purification of MOS). As another example, in some cases, biologically relevant materials can include cells in addition to the refined tissue sample (e.g., biopsy material). In the unpolymerized mixture, the refined tissue sample and the additional biologically relevant material can be present as a uniform mixture or as a distributed mixture (e.g., on one half or another portion of the MOS, such as just in the core or just in the outer region of the formed MOS). In some cases, the additional biologically relevant material within the unpolymerized material can be suspended with the refined tissue sample in suspension (e.g., prior to polymerization of the droplet forming the MOS).

In some cases, the biologically relevant material that optionally can be included with the refined tissue sample (e.g., biopsy) may contain a number of cell types, including preadipocytes, mesenchymal stem cells (MSCs), endothelial progenitor cells, T cells, B cells, mast cells, and/or adipose tissue macrophages, as well as small blood vessels or microvascular fragments found within the stromal vascular fraction, and possibly bone components.

In general, the refined tissue sample (e.g., biopsy) material that is included in the MM MOS described herein is from a bone marrow sample from a mammal (e.g., a human or other mammal having MM, such as a mouse, rat, or rabbit), typically taken by biopsy. These tissues and the resulting refined cells can be primary cells taken from a patient biopsy (e.g., by a needle biopsy. The refined cells can be incorporated into a MM MOS described herein, and can include cells that are commonly found in bone marrow. In that regard, exemplary cells that may be incorporated into MM MOS include plasma cells (normal plasma cells and cancerous plasma cells), stromal cells, hematopoietic stem cells, monocytes, macrophages, dendritic cells, T cells, B cells, and NK cells. The tissue can be refined using any suitable technique, including (without limitation) those described herein.

Moreover, it has now been discovered that in some cases, the preparation and use of MOS generated from MM bone marrow biopsies can be facilitated by (1) using about 50 to about 200 cells (e.g., about 50 to 100 cells, about 75 to about 125 cells, about 100 to about 150 cells, about 100 to about 200 cells, or about 100 cells) per MOS, (2) increasing the concentration of added MATRIGEL® to about 80-100% (e.g., about 80 to about 85%, about 85 to about 90%, about 90 to about 95%, about 95 to about 100%, about 80 to about 90%, or about 90 to about 100%), and/or (3) using ultra low binding plates. Without being bound by a particular mechanism, using relatively higher numbers of cells can increase the likelihood that the full complement of cells from the bone marrow microenvironment (e.g., stromal cells and immune cells) will be included in the MM MOS, which can be important from a drug screening standpoint. In addition, without being bound by a particular mechanism, using MATRIGEL® at a higher concentration can help to retain the MM cells within the MOS, and using low or ultra-low binding plates can reduce the likelihood that the MOS will attach to the bottom of the culture well. In addition, in some cases, the methods disclosed herein for making MM MOS can include adding a small amount of MATRIGEL® (e.g., 1%) to the culture medium rather than to the MOS mix, and/or adding one or more cytokines (e.g., IL-6, GM-CSF, IL-2, B cell activating factor (BAFF), IL-4, or proliferation-inducing ligand (APRIL)—also known as tumor necrosis factor ligand superfamily member 13) to the MATRIGEL®, regardless of whether the MATRIGEL® is added to the MOS mix or to the culture medium. These strategies also may help to retain the MM cells within the MOS and/or to keep the MOS from attaching to the bottom of the culture well.

Given the above, the MM MOS provided herein can have a cell number of about 50 to about 200 cells per MOS, with MM plasma cells, stromal cells, and immune cells included in the MOS cell population. In some cases, the ratio of MM cells to stromal cells in a MOS can be about 1:4 or less (e.g., about 1:4, about 1:5, or about 1:6). In some cases, the ratio of immune cells (e.g., macrophages) to MM cells in a MOS can be about 1:50 to about 1:200 (e.g., about 1:100). The number of MM cells in a MOS cell population can be assessed using, for example, flow cytometry targeting CD19^{neg}, CD38^{pos}, CD56^{pos} (and in some cases also CD138^{pos}) myeloma cells, and using live and/or dead dye to indicate viability of the targeted myeloma cells. The number of other types of cells in a MOS cell population can be determined in a similar fashion. For example, the number of stromal cells in a MOS cell population can be assessed using, for example, flow cytometry targeting CD3^{neg}, CD19^{neg}, CD20^{neg}, CD56^{neg}, CD45^{neg}, CD31^{neg}, ALP^{pos} osteoblasts and/or CD3^{neg}, CD19^{neg}, CD20^{neg}, CD56^{neg}, CD11b^{pos}, CD14^{pos}, RANK^{pos} osteoclasts, and using live and/or dead dye to indicate viability of the targeted stromal cells. The number of immune cells in a MOS cell population can be assessed using, for example, flow cytometry targeting CD3^{pos}, CD4^{pos} or CD8^{pos} T cells, CD3^{neg}, CD56^{pos} NK cells, CD3^{neg}, CD19^{pos} B cells, and/or CD11b^{pos} monocytic cells, and using live and/or dead dye to indicate viability of the targeted immune cells.

Once formed, the MM MOS can be cryopreserved and/or cultured. In general, cultured MOS can be maintained in suspension, either static (e.g., in a well, vial, or other suitable container) or in motion (e.g., rolling or agitated). The MOS can be cultured using any appropriate techniques. Exemplary techniques can be found in, without limitation, Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022, all of which are incorporated herein by reference in their entirety.

In some cases, MM MOS can be produced by forming a droplet of the unpolymerized mixture (in some cases, a chilled mixture) of a refined tissue sample and a fluid matrix material in an immiscible material, such as a fluid hydrophobic material (e.g., oil). For example, a MOS may be formed by combining a stream of unpolymerized material with one or more streams of the immiscible material to form a droplet. The density of the cells present in the droplet can be determined by dilution of the refined material (e.g., cells) in the unpolymerized material. The size of the MOS may correlate to the size of the droplet formed. In general, the MOS is a spherical structure having a stable geometry.

The methods for making MM MOS described herein can employ, unless otherwise indicated, techniques of cell biology, cell culture, molecular biology, transgenic biology, microbiology, recombinant DNA, and immunology. Examples of such techniques are described elsewhere. See, e.g., Molecular Cloning A Laboratory Manual (1989), 2nd Ed., Sambrook, Fritsch, and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, Gait, ed., 1984; Nucleic Acid Hybridization, Hames and Higgins, eds., 1984; Transcription and Translation, Hames and Higgins, eds., 1984; Culture of Animal Cells, Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, Miller and Calos, eds., Cold Spring Harbor Laboratory, 1987; Methods in Enzymology, Vols. 154 and 155, Wu et al., eds., Academic Press Inc., N.Y.; Immunochemical Methods in Cell and Molecular Biology, Mayer and Walker, eds., Academic Press, London, 1987; and Handbook of Experimental Immunology, Volumes I-IV, Weir and Blackwell, eds., 1986.

As mentioned above, the tissue (e.g., biopsy) sample used to form the MM MOS (e.g., the refined sample) can be derived from bone marrow obtained from a mammal having MM. Typically, MOS contain multiple cell types that are resident in the tissue of origin. Thus, the tissue used in the MM MOS provided herein can include cells of the immune system, such as T lymphocytes, B lymphocytes, macrophages, NK cells, monocytic cells, and dendritic cells. The cells can include stem cells, progenitor cells, or somatic cells. The cells may be obtained directly from the subject without intermediate steps of subculture, or they may first undergo an intermediate culturing step to produce a primary culture. Any suitable methods for harvesting cells from biological tissues and/or cell containing fluids can be used. For example, suitable techniques used to obtain cells from biological tissues include those described by Mahesparan, Acta Neuropathol (1999) 97:231-239.

In general, the collected cell types (e.g., within a biopsy or aspirate) can be refined before forming the MOS. For example, cells in a bone marrow aspirate can be refined to isolate for mononuclear cells. Separation of the cell types from one another may be accomplished using any suitable method. For example, the cells in a bone marrow aspirate can be filtered (e.g., through a 70-100 μm strainer) and then subjected to Ficoll gradient purification to separate the mononuclear cells from non-mononuclear cells (e.g., red blood cells, granulocytes, and/or platelets) in the aspirate.

In some cases, the refined tissue can be treated to remove dead/dying cells and/or cell debris. The removal of such dead and/or dying cells can be accomplished using any appropriate means, such as beads and/or antibody methods. For example, since phosphatidylserine can be redistributed from the inner to outer plasma membrane leaflet in apoptotic or dead cells, the use of Annexin V-Biotin binding followed by binding of the biotin to streptavidin magnetic beads can enable the separation of apoptotic cells from living cells. Removal of cell debris can be achieved by techniques such as, for example, filtration.

The refined cells can be suspended in a carrier material prior to being combined with a fluid matrix material. Alternatively, the fluid matrix material can be referred to as a carrier material. In some cases, the carrier material can be a material that has a viscosity level that delays sedimentation of cells in a cell suspension prior to polymerization and formation of the MOS. In such cases, the carrier material can have sufficient viscosity to allow the cells of the refined tissue sample to remain suspended in the suspension until polymerization. The viscosity required to achieve this can be optimized by monitoring the sedimentation rate at various viscosities and selecting a viscosity that gives an appropriate sedimentation rate for the expected time delay between loading the cell suspension into the apparatus that will form the MOS by polymerizing droplets of the unpolymerized material that includes the cells. In some cases, the unpolymerized material can be flowed or agitated by the apparatus (e.g., when lower viscosity materials are used), to keep the cells in suspension and/or distributed as desired.

As discussed above, in some cases the unpolymerized mixture that includes the refined tissue sample and the fluid matrix material can contain one or more other components, such as biologically relevant materials. Biologically relevant material that can be included can include, without limitation, patient-derived serum or plasma, an extracellular matrix protein (e.g., fibronectin), a drug (e.g., a small molecule), a peptide, or an antibody (e.g., to modulate any of cell survival, proliferation or differentiation), an inhibitor of a particular cellular function, and combinations thereof. Such biologically relevant materials may be used, for example, to increase cell viability by reducing cell death and/or activation of cell growth/replication or to otherwise mimic the in vivo environment. The biologically relevant materials can include or may mimic one or more of the following components: serum, interleukins, chemokines, growth factors, glucose, physiological salts, amino acids, and hormones. The biologically relevant materials can, in some cases, supplement one or more agents in the fluid matrix material. In some cases, the fluid matrix material can be a synthetic gel (hydrogel), and can be supplemented with one or more biologically relevant materials. In some cases, the fluid matrix can be a natural gel. Thus, the gel may include one or more extracellular matrix components such as collagen, fibrinogen, laminin, fibronectin, vitronectin, hyaluronic acid, fibrin, alginate, agarose, and/or chitosan. For example, MATRIGEL® includes bioactive polymers that are important for cell viability, proliferation, development and migration. In some cases, the matrix material can be a gel that includes collagen type 1, such as collagen type 1 obtained from rat tails. The gel can be a pure collagen type 1 gel or can be one that contains collagen type 1 in addition to other components, such as other extracellular matrix proteins. In some cases, the fluid matrix can be a synthetic gel that does not occur in nature. Examples of synthetic gels include gels derived from polyethylene glycol (PEG), polyhydroxyethyl methacrylate (PHEMA), polyvinyl alcohol (PVA), or polyethylene oxide (PEO).

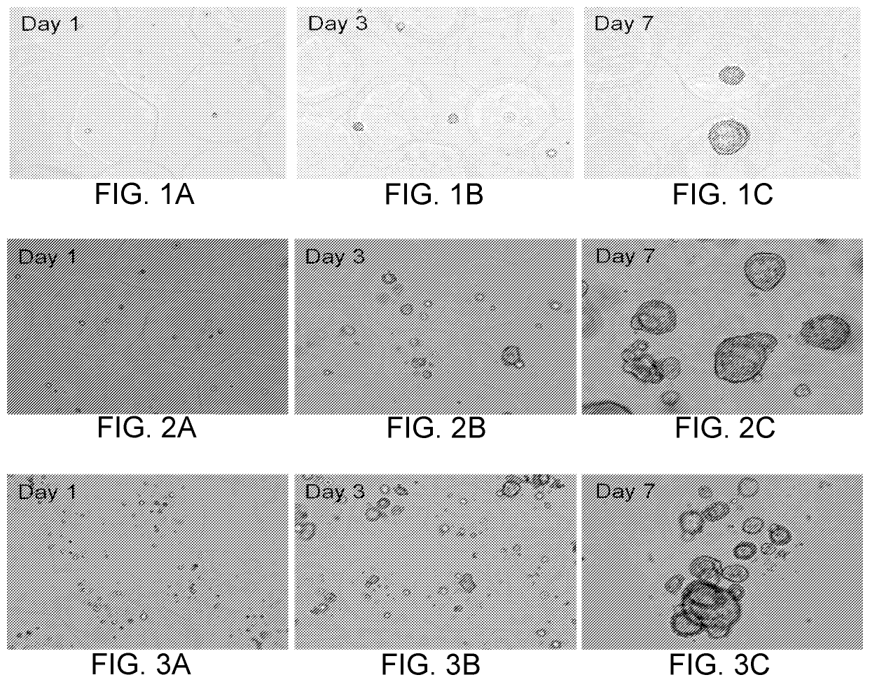

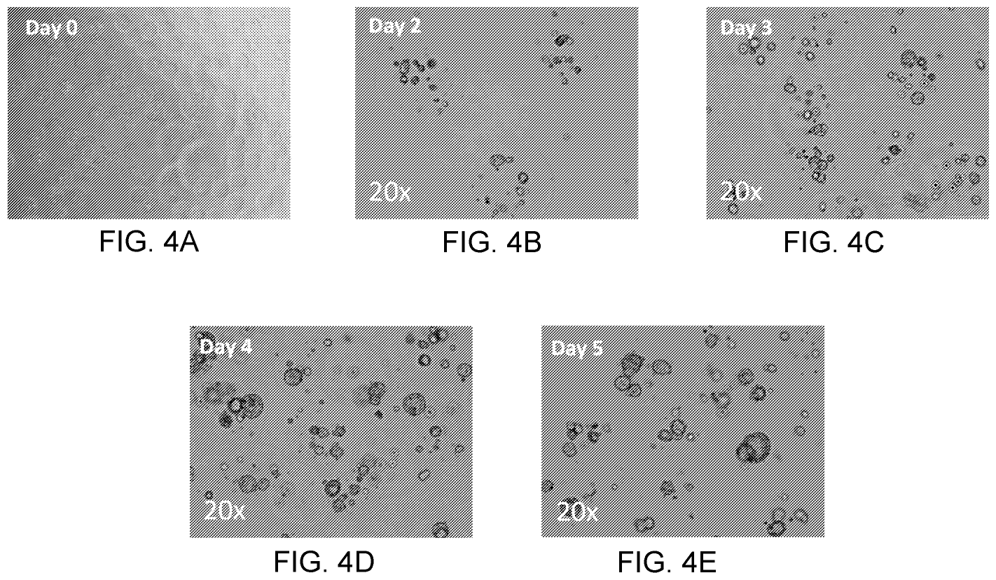

Images showing PDMOs are presented in FIGS. 1A-1C, 2A-2C, 3A-3C, and 4A-4E. For example, FIGS. 1A-1C include representative images of MOS formed having a single cell per MOS. As shown, the MOS are all about the same size, having a diameter of about 300 μm. After three days in culture (center panel), the cells had expanded in size, in some cases doubling and/or growing. By seven days in culture (right panel), the cells had doubled multiple times, showing clusters or masses of cells. The MOS shown in FIGS. 2A-2C were formed from five cells per MOS, while the MOS shown in FIGS. 3A-3C were formed from 20 cells per MOS. In FIGS. 4A-4E, the MOS are shown immediately after formation and cultured for five days, in which nearly identical MOS (e.g., having the same diameter) each include 10 cells per MOS. In FIG. 4A, the MOS are shown immediately after forming, still surrounded by the immiscible fluid (in this case, oil) at day 0. The MOS were removed from the immiscible fluid, washed, and cultured for five days. FIGS. 4B, 4C, 4D, and 4E show the MOS after two, three, four, and five days, respectively. These images show that the refined tissue (cells) from the biopsy within the MOS were viable and growing within nearly all of the MOS at comparable rates. As will be described in greater detail herein, these MOS can be formed in large numbers from even a single, average-sized biopsy, and may result in hundreds or even thousands (e.g., about 500, about 750, about 1000, about 2000, about 5000, about 10,000, about 100 to about 600, about 600 to about 1000, more than about 1000, or more than 10,000) of MOS that include a significant number of viable cells, allowing multiple rapid assays to be performed in parallel.

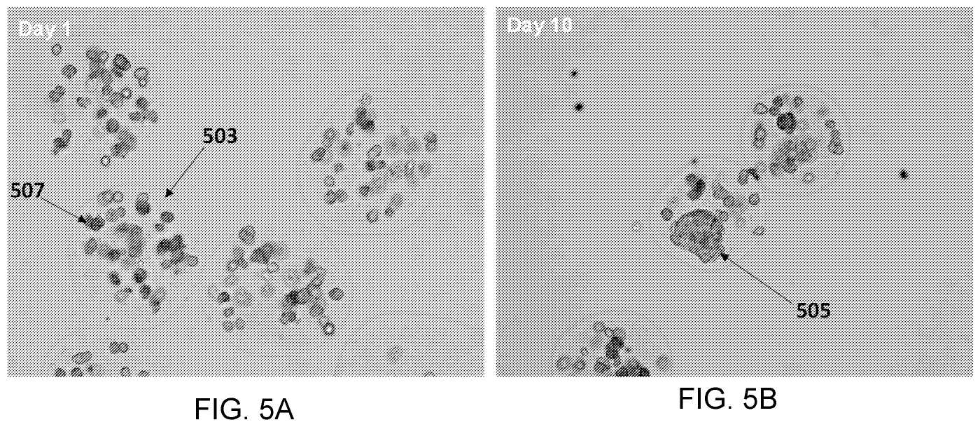

FIGS. 5A and 5B illustrate an example of MOS formed as described herein from a biopsy of mouse liver, showing mouse hepatocytes distributed within a polymerized fluid matrix material (in this example, MATRIGEL®). Each MOS included the polymerized matrix material 503 formed into a sphere having a diameter of about 300 μm, in which a set number of hepatocytes 507 were dispersed. In FIG. 5A, the MOS are shown one day after biopsy, dissociation or refining of the tissue, and forming of the MOS. These MOS were then cultured for 10 days, during which time the hepatocytes remained viable and grew, in many cases doubling multiple times to form structures 505 as shown in FIG. 5B.

MOS generally can include dissociated or refined biopsy tissue (e.g., cells) in a fixed or known number or concentration of cells (e.g., cells/ml or cells/mm³) within the MOS. As noted above, this matrix material can include natural polymers, such as one or more of alginate, agarose, hyaluronic acid, collagen, gelatin, fibrin, and elastin, or can include a synthetic polymer, such as one or more of PEG and polyacrylamide. Both organic and inorganic synthetic polymers can be used.

In some cases, the number of cells initially included in the MOS can be selected from between 1 cell up to several hundred cells. In particular, in some assays (e.g., drug toxicity assays) it may be beneficial to include about 1 to about 75 cells or about 1 to about 50 cells (that is, relatively lower numbers of cells). The number of cells per MOS can be set or selected by the technician generating the MOS. In some cases, as described below, an apparatus for generating MOS can include one or more controls to set the number of cells from the primary tissue that will be included in each MOS. The number of cells can be chosen or set based on how the MOS are intended to be used. For example, MOS having very low number of cells (e.g., 1 cell per MOS or 1 to 5 cells per MOS) can be particularly suitable for studying clonal diversity (e.g., for tumor heterogeneity). Since each MOS grows from a single cell, one can observe which clones are drug resistant, and these specific MOS can then be examined (e.g., by genomic sequencing) to determine the genomic (mutation) diversity related to the particular clone. A low to moderate number of cells per MOS (e.g., about 3 to about 30 cells, about 5 to about 30 cells, about 5 to about 25 cells, about 5 to about 20 cells, or about 10 to about 25 cells) can be particularly useful for rapid drug testing, including toxicity testing since these MOS typically grow quickly. A larger number of cells per MOS (e.g., about 20 to about 100 cells, about 30 to about 100 cells, about 40 to about 100 cells, or more than 50 cells) can be particularly suitable for mimicking tissue composition in each MOS, as the MOS may contain different lineages—potentially including epithelial (e.g., cancer) and mesenchymal (e.g., stromal, immune, or blood vessel) cells. It is noted that for MM MOS, including a relatively large number of cells per MOS (e.g., about 50 to about 150 cells per MOS) can provide each MOS with different cell types, which can act to maintain the MM cells.

MOS can be formed in any appropriate size, which may be matched to the number of cells to be included. For example, the size of a MOA may be relatively small, having a diameter of about 20 μm to about 500 μm (e.g., about 50 μm or about 100 μm on average, or about 100 μm to about 200 μm). In some cases, the size of a MOS can be about 300 μm, in which case about 10 to about 50 cells (e.g., about 10 to about 30 cells) can be included in each MOS. The number of cells and the size can be varied and/or may be controlled. In some cases, the number of cells and/or the size of the MOS can be set by one or more controls on the apparatus used to forming the MOS. For example, the size of the MOS and/or the density of cells within the MOS can be adjusted by adjusting the flow rates and/or the concentration of the refined tissue sample (e.g., cells from a biopsy).

As shown in FIGS. 1, 2, 3, 4A-4E, and 5A-5B, even after culturing the MOS described herein, viable and healthy cells were observed through the entire volume of the MOS. The size of the MOS and/or the number of cells to be included in the MOS can be selected based on how the MOS are expected or intended to be used. For example, in variations in which the MOS are to be used to examine relationships between cells of the biopsied material, the MOS can be formed to include multiple cells (e.g., multiple types of cells or multiple cells of a single type), and can be cultured for extended periods of time (e.g., up to one week or more).

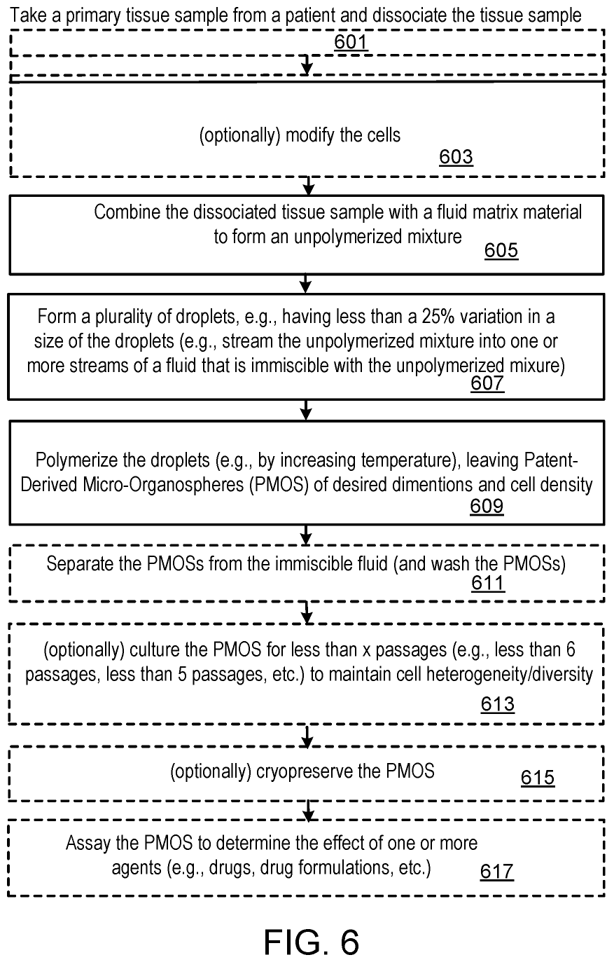

In some cases, PDMOs can be generated by combining a dissociated or refined tissue sample (e.g., a biopsy sample) with a fluid matrix that can polymerized in a controlled manner to form MOS. FIG. 6 illustrates a representative method for forming PDMOs. Optionally, the method can include taking a sample from a mammal (e.g., a human having MM), such as taking a biopsy from a patient tissue 601. The biopsy can be obtained using, for example, a biopsy needle or punch. In some cases, for example, a bone marrow biopsy can be taken with a 14-gauge, 16-gauge, 18-gauge, 20-gauge, or 22-gauge needle that is inserted into a mammal to remove the biopsy. After removing the tissue from the mammal, the tissue can be processed to dissociate the cells and other material (e.g., in the case of a solid tumor sample) or to refine the sample by removing certain types of cells (e.g., in the case of a bone marrow aspirate), using mechanical and/or chemical techniques. The dissociated or refined cells can immediately be used to form the PDMOs, as described herein, or in some cases, all or some of the cells can be modified, such as by genetically modifying the cells 603 (e.g., using transfection or electroporation to introduce one or more proteins or nucleic acids that will lead to a genetic modification).

With further reference to FIG. 6, the dissociated or refined tissue sample from the biopsy material can be combined with a fluid (e.g., a liquid) matrix material to form an unpolymerized mixture 605. This unpolymerized mixture can be held in an unpolymerized state, so that the cells from the dissociated or refined tissue remain suspended within the mixture. In some cases, the cells can remain suspected and unpolymerized by keeping them chilled (e.g., at room temperature or below, such as a temperature of 1° C. to 25° C.).

The unpolymerized mixture can then be dispensed as droplets into an immiscible material, such as an oil, in a manner that controls the formation of the size of the droplets and therefore the size of the PDMOs that are formed 607. For example, uniformly sized droplets can be formed by combining a stream of the unpolymerized material into one or more (e.g., two converging) streams of the immiscible material (e.g., oil) so that the flow rates and/or pressures of the two streams determine how droplets of the unpolymerized material are formed as they intersect the immiscible material. The droplets can be polymerized 609 to form the PDMOs in the immiscible material. In some cases, the immiscible material can be heated or warmed to a temperature that causes the unpolymerized mixture (e.g., the fluid matrix material in the unpolymerized material) to polymerize. Once formed, the PDMOs can be separated from the immiscible fluid. For example, the PDMOs can be washed to remove the immiscible fluid 611, and placed in a culture media to allow the cells within the PDMOs to grow. The PDMOs can be cultured for any desired time, or they can be cryopreserved, or they can be assayed immediately. In some cases, the PDMOs can be cultured for a period of time (e.g., from 1 to 3 days, 1 to 4 days, 1 to 5 days, 1 to 6 days, 1 to 7 days, 1 to 8 days, 1 to 9 days, 1 to 10 days, 1 to 11 days, or 1 to 14 days). Culturing the PDMOs can allow the cells derived from the biopsy tissue to grow and/or divide (e.g., double) for up to five or six passages. After culturing, the cells can be cryopreserved 615 and/or assayed 617. Examples of assays that can be used also are described herein.

In any of the methods and apparatuses described herein, the MOS can be recovered from the immiscible fluid (e.g., oil) after polymerization. For example, in some cases, the MOS can be recovered by demulsification and/or de-emulsification, such as by forming emulsified droplets and recovering the MOS after the droplets are formed to remove any oil (and other contaminants). This can allow the cells to grow within the polymerized droplet (the MOS) without being inhibited by the immiscible fluid.

Although the methods and apparatuses described herein illustrate methods of forming the plurality of droplets, and thus the plurality of MOS, by streaming the unpolymerized mixture into one or more streams of immiscible fluid (such as an oil or other hydrophobic material), in some cases the droplets can be formed by other methods that may allow the size of the droplet to be controlled as described herein. For example, in some cases, the droplets can be formed by printing (e.g., by printing droplets onto a surface). This can reduce or eliminate the need for an additional recovery step of emulsification/de-emulsification. For example, the droplets may be printed onto a surface, such as a flat or shaped surface, and polymerized. In any of these variations, the droplets can be dispensed using pressure, sound, charge, or any other appropriate means. In some cases, the droplets can be formed using an automatic dispenser (e.g., a pipetting device) adapted to release a small amount of the unpolymerized mixture onto a surface, into the air, and/or into a liquid medium, such as an immiscible fluid).

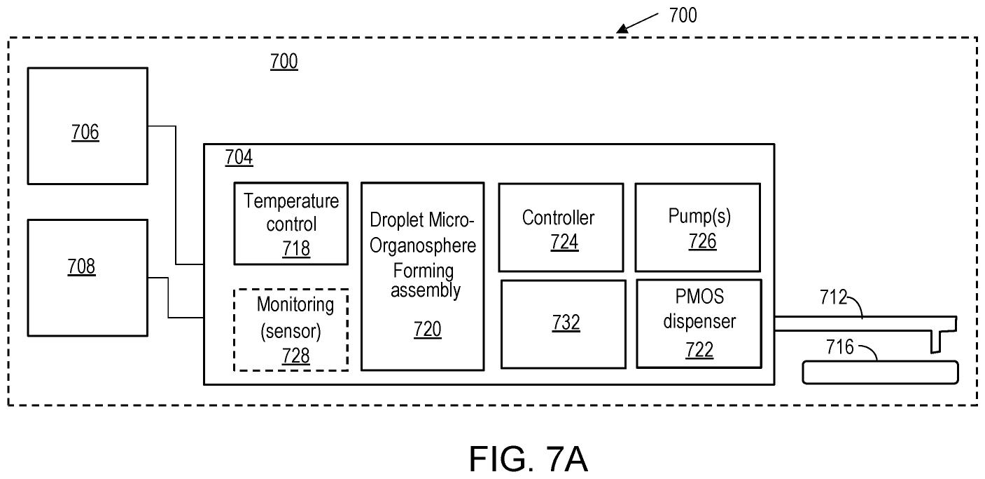

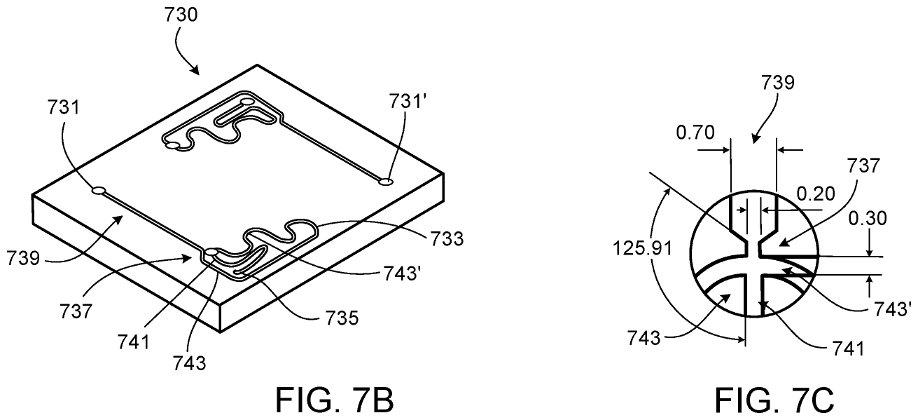

A method for forming PDMOs can be automated and/or performed using one or more apparatuses. In particular, a method of forming PDMOs can be performed by an apparatus that allows for the selection and/or control of the size of the MOS, and therefore the density of the number of cells. For example, FIG. 7A illustrates one example of an apparatus 700 for forming PDMOs as described herein.

As depicted in FIG. 7A, the apparatus can include an input for inputting either the unpolymerized mixture of the dissociated or refined tissue sample and a fluid matrix material (already combined), or an input for separately receiving the dissociated or refined tissue sample (e.g., in a holding solution) and a fluid matrix material. In some cases, the apparatus can include a holding chamber 706 for retaining the unpolymerized mixture, and/or holding chambers (not shown) for retaining the dissociated or refined tissue (e.g., biopsy) sample and holding the fluid matrix material. Any or all of these holding chambers can be pressurized to control and/or speed up fluid flow out of the chambers and into the device. The apparatus can either receive the unpolymerized mixture, or the apparatus can receive the components and mix them. In some cases, the apparatus can control the concentration of the cells in the unpolymerized mixture, and can dilute the mixture (e.g., by adding additional fluid matrix material to achieve a desired density). For example, the apparatus can include a sensor (e.g., an optical reader) for reading the density (e.g., the optical density) of the cells in the unpolymerized mixture. The sensor also can be coupled to the controller 724, which can automatically or semi-automatically (e.g., by indicating to a user) control the dilution of the cells in the unpolymerized mixture. The apparatus also can include a port for receiving the unpolymerized mixture. The port can include a valve or be coupled to a valve, and the valve can be controlled by the controller 724 or by a separate controller.

The apparatus 700 can include a chamber 708 and/or port for holding and/or receiving the immiscible fluid. In some cases, the immiscible fluid can be held in a pressurized chamber so that the flow rate can be controlled. Any of the pressurized chambers can be controlled by the controller 724, which may use one or more pumps 726 to control the pressure and therefore the flow through the apparatus. One or more pressure and/or flow sensors can be included in the system to monitor the flow through the device.

In FIG. 7A, the entire apparatus 700 can be enclosed in a housing 702, or a portion of the apparatus 704 can be enclosed in a housing. In some cases, the housing can include one or more openings or access portions on the device, e.g., for adding the immiscible fluid and/or the unpolymerized mixture.

As noted above, apparatus 700 also can include one or more sensors 728 for monitoring all or key portions of the manufacturing process. In some cases, the sensor(s) can include one or more optical sensors, mechanical sensors, voltage and/or resistance (or capacitance or inductance) sensors, or force sensors. The sensors can be used to monitor the ongoing operation of the assembly, including the formation of the PDMOs. The apparatus 700 also can include one or more thermal/temperature regulators 718 for controlling the temperature of the immiscible fluid and/or the unpolymerized mixture and/or the fluid matrix material.

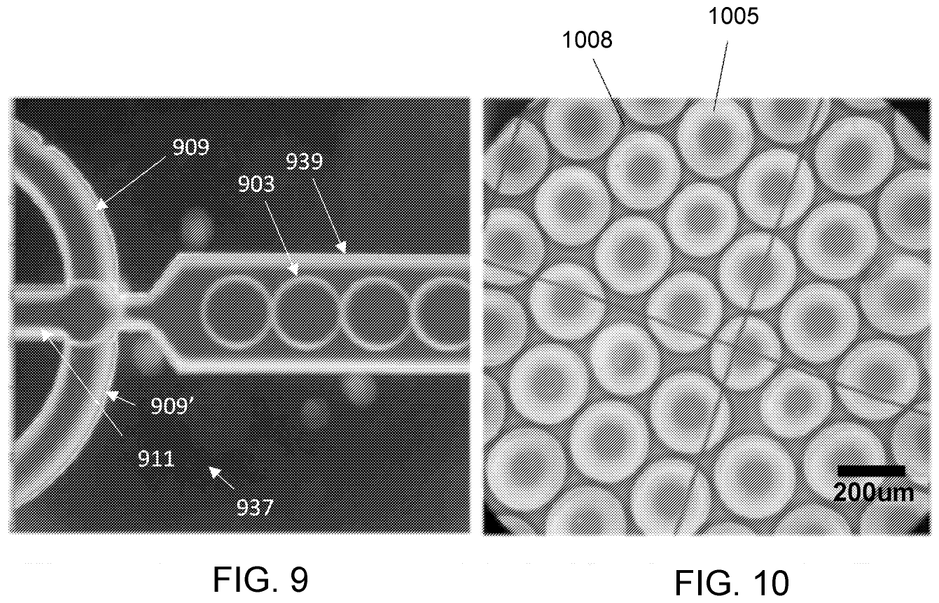

Apparatus 700 also can include one or more droplet forming assemblies 720 that can be monitored (e.g., using one or more sensors) as is illustrated in FIGS. 7C and 9 and discussed below. The droplet MOS forming assembly can include or be coupled with a dispenser (e.g., a PMOS dispenser) 722. The dispenser can dispense droplets into, for example, a multi-well plate 716.

In general, the droplet MOS forming assembly 720 can include one or more microfluidic chips 730 or structures that form and control the streams of unpolymerized mixture and form the actual droplets. FIG. 7B illustrates one example of a microfluidic chip for forming PDMOs 730. In FIG. 7B, the chip 730 includes a pair of parallel structures for forming MOS. FIG. 7C illustrates a droplet-forming region of the microfluidic chip for forming PMOSs, including an unpolymerized channel outlet 741 that opens (in this example, as a right angle) a “+” junction or region of intersection 737 to the channel outlet 741 and the immiscible fluid outlet(s), 743, 743′. In some cases, the input from the immiscible fluid channel(s) can be at an angle relative to the angle (and point of intersection) with the unpolymerized material. In FIG. 7C, as in all figures in this description showing dimensions, the dimensions shown are exemplary only, and are not intended to be limiting, unless they otherwise specify.

In FIG. 7A, the microfluidics chip 730 includes an inlet (input port) 733 for the immiscible fluid into the chip (e.g., from the inlet port or storage chamber shown in FIG. 7A). A second inlet port 735 into the chip can be configured to receive the unpolymerized material and transport it down a semi-tortious path to the junction region. Similarly, the inlet port for the immiscible fluid can be securely coupled to the outlet from the immiscible fluid chamber or inlet, described above.

The inlet port 735 for the unpolymerized material into the chip can be coupled through a delivery pathway 741 connecting the inlet to the junction region (as shown in FIG. 7C). Similarly, the inlet 733 for the immiscible fluid can connect two or more connecting paths 743, 743′ to the junction region 737. A channel leaving the junction region 737 can pass the formed MOS (in the immiscible fluid) down the channel to an outlet 731 that can connect to a dispenser for dispensing the MOS into one or more chambers (e.g., for culture and/or assaying).

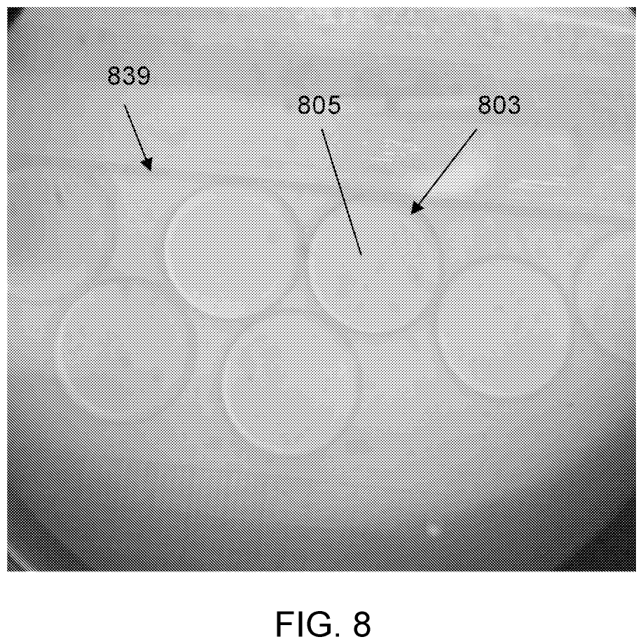

In the example shown in FIGS. 7B and 7C the formed droplets, which may become MOS once polymerized, can be transmitted down a long, temperature-controlled microfluidics environment prior to being dispensed from the apparatus. For example, FIG. 8 illustrates one example of a channel region 839 (e.g., element 739 in FIG. 7B) that is shown transparent, containing a plurality of MOS 803, each containing a predetermined number of cells 805. It is to be noted that in any of the microfluidic chips or devices described herein, the channels can be coated. For example, the channels of a microfluidic device can be coated with a hydrophobic material.

FIG. 9 depicts a junction region 937 that is shaped as described above, so that the channel carrying the unpolymerized mixture 911 intersects one or more (e.g., two) channels 909 carrying a fluid, such as an oil, that is immiscible with the unpolymerized mixture. As the unpolymerized mixture is pressurized to flow at first rate out of the first channel 911, the flowing immiscible fluid in the intersecting channels, 909, 909′, permit a predefined amount of the unpolymerized mixture to pass before pinching it off to form a droplet 903 that is passed into the outlet channel 939. Thus, in some variations, a minced (e.g., dissociated) clinical (e.g., biopsy or resected) sample of tissue having a size that is, for example, less than 1 mm in diameter, can be mixed with a temperature-sensitive gel (e.g., MATRIGEL® at 4° C.) to form the unpolymerized mixture. The unpolymerized mixture can be placed into the microfluidic device that generates droplets (e.g., water-in-oil droplets) that are uniform in volume and material composition. Simultaneously, the dissociated tumor cells can be partitioned into these droplets. The gel in the unpolymerized material can solidify upon heating (e.g., at 37° C.), and the resulting PDMOs can be formed. In some cases, this method can be used to produce at least 10,000 (e.g., at least 20,000, at least 30,000, at least 40,000, at least 50,000, at least 60,000, at least 70,000, at least 80,000, at least 90,000, or at least 100,000) uniform droplets (PDMOs) from the tissue (e.g., biopsy material). These PDMOs are compatible with traditional 3D cell culture techniques. FIG. 10 illustrates a plurality of PDMOs 1005 formed as described above, suspended in immiscible material 1008 (e.g., oil).

In the exemplary microfluidics chip illustrated above, the junction is shown as a T- or X-junction in which the flow focusing of the microfluidics forms the controllable size of the MOS. In some cases, rather than a microfluidics chip, the droplets can be formed by robotic micro-pipetting into an immiscible fluid and/or onto a solid or gel substrate, for example. Alternatively, in some cases, the droplets of unpolymerized material can be formed in the requisite dimensions and reproducibility by micro-capillary generation. Other examples of techniques that can alternatively be used for forming the MOS in the specified size range and reproducibility from the unpolymerized material include, without limitation, colloid manipulation, e.g., via external forces such as acoustics, magnetics, inertial, electrowetting, or gravitation.

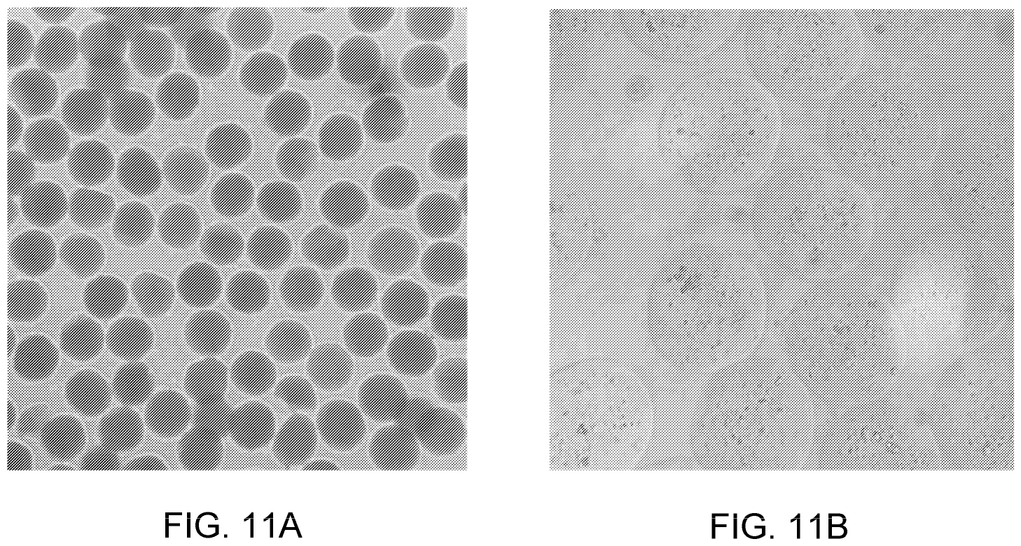

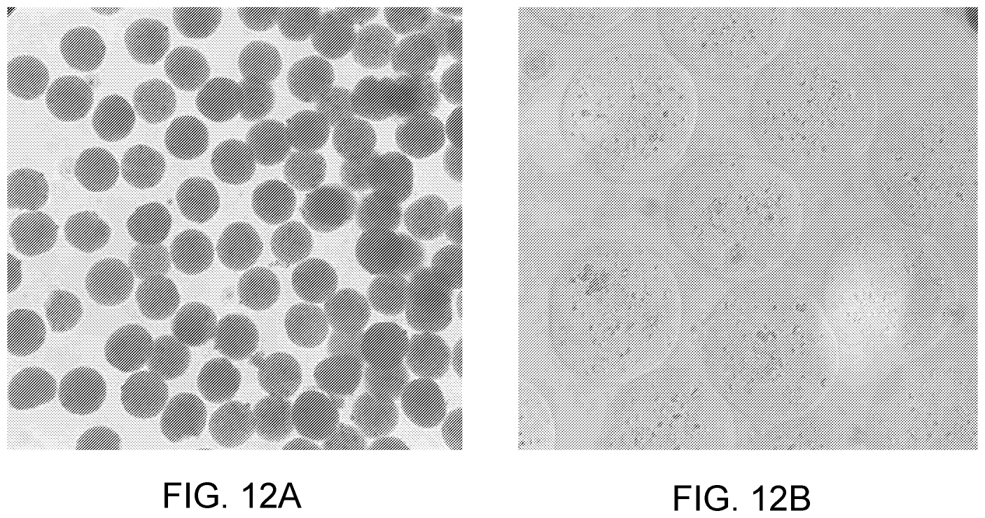

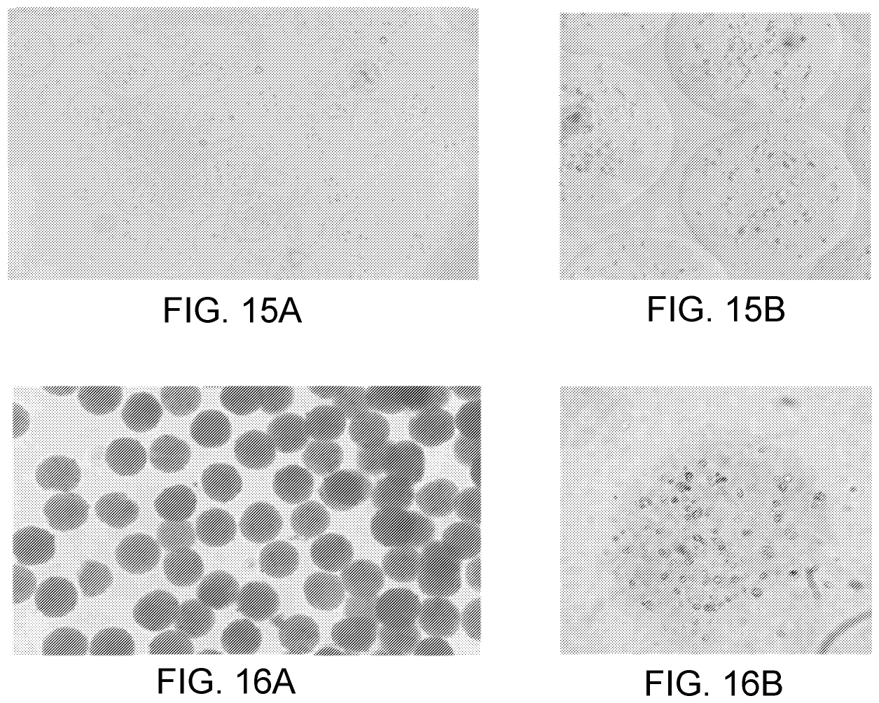

FIGS. 11A and 11B shows examples of PDMOs in oil formed as described above. The cells within these MOS, derived from a single biopsy sample, are viable, as seen by vital dye staining (FIGS. 15A-15B and 16A-16B). For example, FIGS. 12A and 12B illustrate MOS containing tumor cells (similar to those shown in FIGS. 11A and 11B) that can be washed to remove the immiscible material (e.g., oil). The immiscible material can be removed relatively quickly after forming the MOS in order to prevent harm to the cells within the MOS.

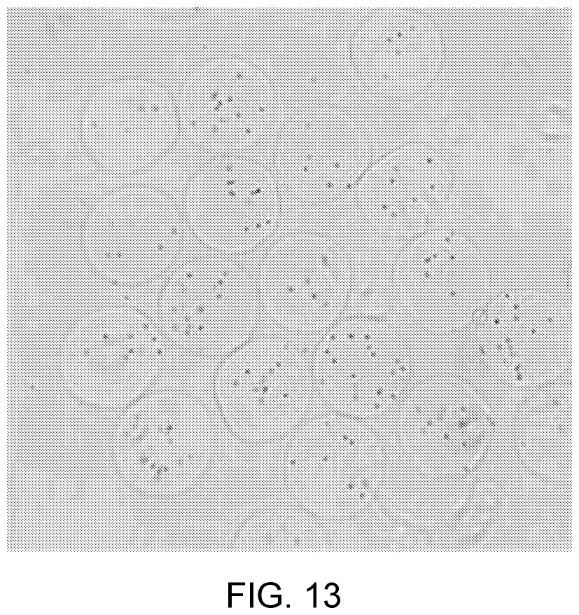

In these examples, the gel droplets can be recovered from the oil phase and resuspended in, for example, PBS via PFO (perfluoro octanol). Centrifugation can be used to separate the immiscible fluid from the MOS. The MOS can then be grown, as shown in FIGS. 1, 2, 3, 4A-4E, and 13. This important, as drug screening must be performed on viable and growing primary tumor cells that retain their properties from patient tumors to predict patient outcomes. The high number and uniformity of these MOS makes screening both possible and reliable, as will be described below.

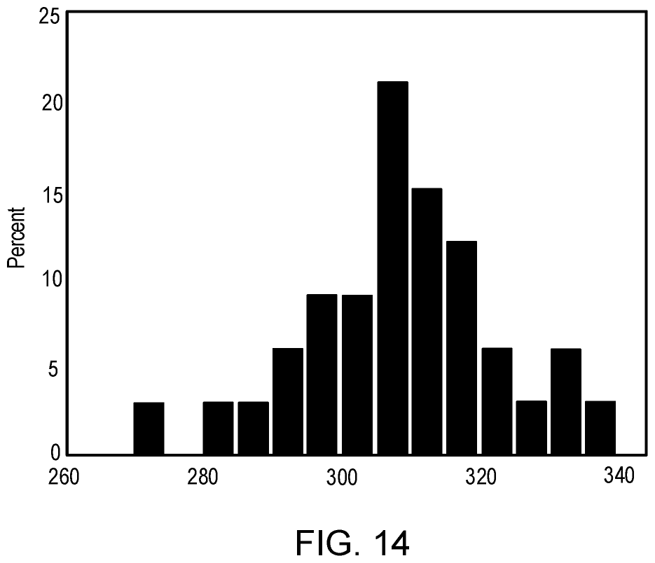

In general, the MOS provided herein are highly uniform in diameter, and can have a very low size (e.g., diameter) variance. This is illustrated, for example, in FIG. 14, which shows a representative distribution of droplet diameter sizes.

FIGS. 15A and 15B show another population of MOS formed as described herein. In FIGS. 16A and 16B, these MOS were stained with Trypan blue (arrowheads) to show that they are alive. In some cases, MOS formed as droplets as described herein can contain growth-factors and matrix components to mimic the biological environment from which the cells in the MOS. Mammalian samples (e.g., patient biopsy samples) typically can be formed into MOS within hours (e.g., about 6 to 12 hours, about 12 to 18 hours, about 18 to 24 hours, about 12 hours, about 18 hours, about 24 hours, or about 48 hours) of acquiring the tissue. Each MOS can contain only a few cells (e.g., as few as 1 cell or about 4 to 6 cells, such as cancer cells when sampling a tumor), or each MOS can contain more cells (e.g., 50 to 150 cells in the case of MM MOS). Methods analogous to those described herein have been shown to generate MOS from multiple types of cancer and non-cancerous tissues, including colon, esophagus, melanoma, uterus, sarcoma, renal, liver, ovary, lung, diaphragm, omentum, mediastinal lung, and breast cancer tissues (see, e.g., the Working Examples of U.S. Publication No. 2021/0285054, which is incorporated herein by reference in its entirety). The MOS can be cultured for any desired period of time, and typically show proliferation and growth in as few as 3 to 4 days. The MOS can be maintained and passaged for months. As will be described in greater detail below, the MOS also can be used to screen drug compositions within as few as 4 to 14 days (e.g., 4 to 6 days, 5 to 7 days, 6 to 8 days, 7 to 10 days, 8 to 12 days, or 10 to 14 days) from taking the tissue (e.g., a biopsy).

The MOS described herein can, at any point after they are formed, be banked, such as by cryopreservation. Tumor MOS can be collected from many different patients and can be used individually or collectively to screen multiple drug formulations to determine toxicity and/or efficacy of particular therapeutic agents. In some cases, non-tumorous cells (healthy tissue) can be biopsied, banded, and/or screened in parallel. Thus, the methods and apparatuses provided herein can allow for high throughput screening. In some variations, the MOS can be formed, allowed to passage twice (e.g., for two doublings), and cryopreserved. Again, normal, healthy tissue also can be used to form corresponding MOS to generate hundreds, thousands, or tens of thousands of MOS that can be used for assaying drug effects, drug response, biomarkers, proteomic signals, genomic signals, etc.

It is of particular significance that the MOS survive in a biologically significant manner, allowing them to provide clinically and physiologically relevant data, particularly with respect to drug response. In particular, the MOS provided herein permit tissue extract/biopsy originated cells to grow exceptionally well and provide more representative data, especially as compared to organoids or spheroids. Without being bound by a particular mechanism, this may be because the cells may have a more constrained cell density in the MOS, permitting cells to communicate without inhibiting each other while sharing signals. The MOS also have a very large surface to volume ratio, more readily permitting transmission of growth factors and other signals to penetrate into the MOS (that is, the MOS are less diffusion limited).

The PDMOs (e.g., MM MOS) described herein can be used in a variety of different assays, and in particular can be used to determine drug formulation effects, including toxicity, on MM tissue. As used herein, a drug composition can include any drug, drug dilution, drug formulation, compositions including multiple drugs (e.g., multiple active ingredients), drug formulations, drug forms, drug concentrations, combination therapies, and the like. In some cases, a drug formulation refers to a formulation containing a mixture of a drug and one or more inactive ingredients.

In some cases, drug screening can include applying MOS into all or some wells of a multi-well (e.g., a 96-well) plate. Alternatively, custom plates can be used (e.g., a 10,000 micro-well array can be formed of a 100×100 well grid). The MOS (e.g., gel droplets) can be applied into, or in some cases onto, the multiple microwell arrays and incubated with culture medium. The MOS can be cultured over the course of about 3 to about 14 days. In some cases, on a selected day (e.g., day 5), the wells (e.g., micro-reactors) can be dosed with drug compounds, e.g., based on a set of FDA-approved anticancer drugs, such as drugs for treating MM, to examine the effects of the drug panel. For example, the drugs tested can be based on the National Cancer Institute (Division of Cancer Treatment and Diagnosis) screen, consisting of 147 agents intended to enable cancer research, drug discovery and combination drug studies. In some cases, the drugs tested can include one or more agents selected from immunomodulatory agents (e.g., IMiDs, thalidomide, lenalidomide, and pomalidomide), PIs (e.g., bortezomib, carfilzomib, and ixazomib), monoclonal antibodies (e.g., elotuzumab, daratuzumab, isatuximab, and belantamab), nuclear exporter inhibitors (e.g., selinexor), doxorubicin, panobinostat, and melflufen, steroids (e.g., dexamethasone), alkylating agents (e.g., cyclophosphamide, melphalan, and bendamustine), CAR T cell therapies (e.g., Abecma, and Ide-cel), and bi-specific antibodies. On a subsequent selected day (e.g., day 7), the MOS can be imaged (e.g., via standard fluorescent microscopy) and ranked based on drug response. In some cases, a drug IC₅₀ curve can be generated using flow-based live myeloma cells counts with any appropriate number of drug titrations (e.g., three, four, five, six, seven, eight, nine, ten, three to five, five to seven, or seven to nine titrations) and replicates per titration (e.g., two to four, two to three, three to five, or three to four replicates per titration).

After MOS are treated with one or more potential therapeutic agents, the cells therein can be assessed to determine the level of myeloma cell killing, thereby indicating the efficacy of the tested therapeutic agent(s). Cells can be removed from the MOS using any appropriate technique. For example, the MOS can be heated to melt the base material (e.g., the hydrogel). The cells then can be stained with antibodies specific for markers of the cells to be detected. For example, antibodies against CD19, CD38, and CD58 (and optionally CD138) can be used to stain myeloma cells within a population of cells. The cells also can be stained with a label for live cells, a label for dead cells, or a label for live cells and a label for dead cells. Non-limiting examples of label that can be used to distinguish live cells from dead cells include GHOST DYE™ (CYTEK® Biosciences; Fremont, CA), LIVE/DEAD fixable dead cell stains (ThermoFisher Scientific; Waltham, MA), propidium iodide, Zombie Dyes (BioLegend; San Diego, CA), Phantom Dye (Proteintech; Rosemont, IL), and HORIZON™ dyes (Becton Dickinson; Franklin Lakes, NJ). After labeling, the cells can be subjected to flow cytometry to detect and quantify living and/or dead cells of the targeted type(s). It is to be noted that the above-described assay can be done at the well level, to assess the effect of one or more therapeutic agents on cells within all of the MOS within a well, or the level of an individual MOS.

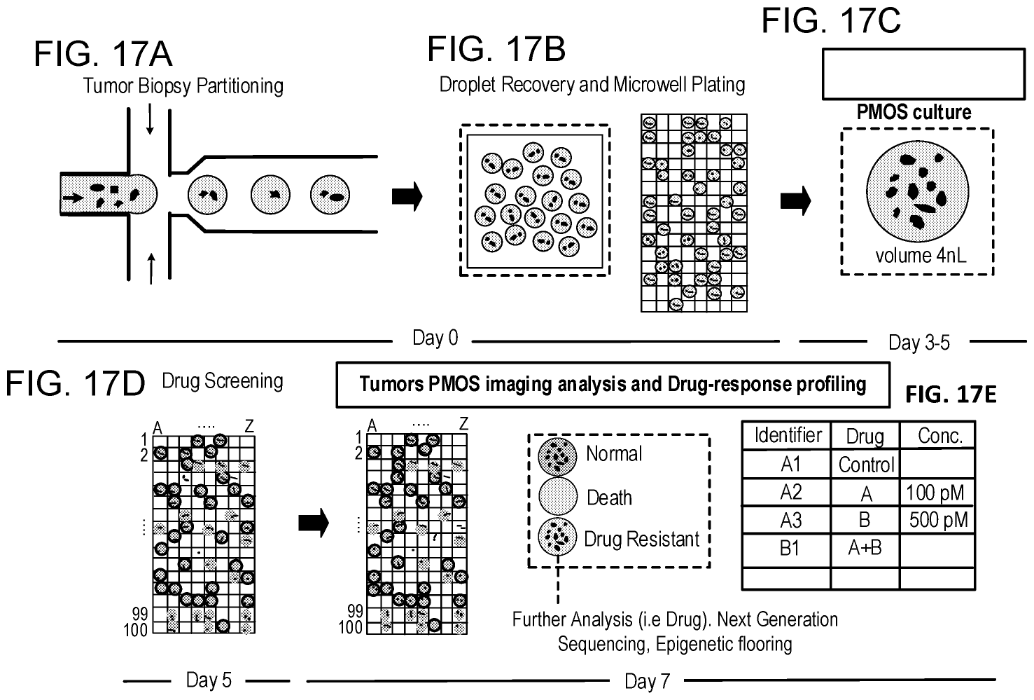

An example of an assay/screening technique is illustrated in FIGS. 17A-17E. In this example, the screening assay can be automated, which can enable repeatable and automated workflow, increasing the number of drugs that can be tested in a screen. In FIG. 17A, a tumor biopsy is taken and a plurality (e.g., >10,000) of MOS are formed as described above (in FIG. 17A, the junction region forming the MOS is illustrated). Thereafter, the MOS can be recovered and washed to remove the immiscible material (e.g., oil) in which they were formed. The MOS then can be plated into one or more microwell plates. As shown in FIG. 17C, the MOS can be cultured for one or more generations (e.g., one or more passages). This is shown occurring from day 0 to day 3, 4, or 5. Thereafter, the MOS can be screened as shown in FIG. 17D, such as by applying drugs to a subset of the replicate wells. Subsequently, as shown in FIG. 17E the cells in the MOS can be imaged and/or automatically or manually scored to identify drug effects (e.g., drug screening and growth profiling).

The workflow illustrated in FIGS. 17A-17E can enable an integrated device to be used for growing, dosing, and/or reviewing MOS. In an exemplary device, freshly biopsied or resected patient tumor samples can be disassociated and seeded into a gel with regents to form MO as described herein. In some cases, a portion of the MOS formed can be cryopreserved. The rest can be recovered and incubated until being seeded into microwell plates for drug testing or screening as described. Growth and viability assays can be performed on the MOS, which may be imaged and tracked. The MOS response to drug treatments, such as IC₅₀, cytotoxicity, and growth curves, can be measured to identify effective therapeutics against a patient's tumor (e.g., MM).

The methods and apparatuses described herein have numerous advantages, including reproducibility. The sample preparation process can be automated by the microfluidic sample partitioning, which can reduce the need for specialized personnel for diagnostic testing and manual pipetting. This can be particularly helpful in a clinical setting. Moreover, this can enable uniformity among signal droplets, increasing assay sensitivity. In addition, these assays can minimize the time required to generate MOS. In some cases, these methods can be used to generate a library of over 100,000 MATRIGEL®-tumor droplets (MOS) in less than about 15 minutes. The methods also are highly scalable, and can be multiplexed to run multiple patient biopsies in parallel.

In addition, the methods described herein are flexible and compatible with other techniques. As a research tool, droplet-based microfluidics is generally compatible with a wide range of hydrogel materials such as agarose, alginate, PEG, and hyaluronic acid, for example. As such, the starting gel composition can readily be modified to accompany and encourage MOS growth. Moreover, the droplet-size can be adjusted by modifying the size of the microfluidic device. Together, these options allow for a large selection of gel material composition and micro-reactor sizes.

The miniaturized assays described herein (using MOS) can maximize the utility of a patient tumor biopsy, enabling more drug compounds to be screened. For example, a 600 μL bone marrow sample from a mammal with MM can be partitioned into about 143,000 individual micro-reactors that are each about 4 nL in volume. By maximizing the tissue sample, multiple experimental replicates can be examined, increasing statistical power. These techniques can allow the inspection of intra-tumor heterogeneity, drug perturbation, and can identify rare cellular events, such as drug resistance. The MOS generally can be compatible with downstream assays, such as single cell RNA transcriptome analysis and epigenetic profiling. In addition, by maximizing the tissue (e.g., biopsy) sample efficiency as provided by the MOS, a portion of the MOS can be banked (e.g., by cryopreservation) for future novel drug assays and/or for confirmation analysis, including genetic screening.

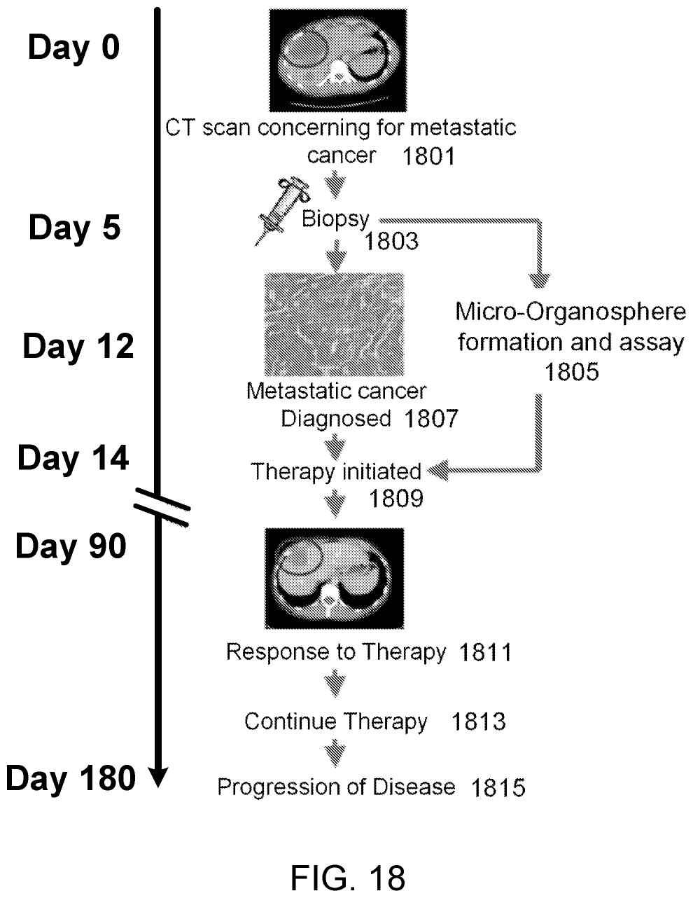

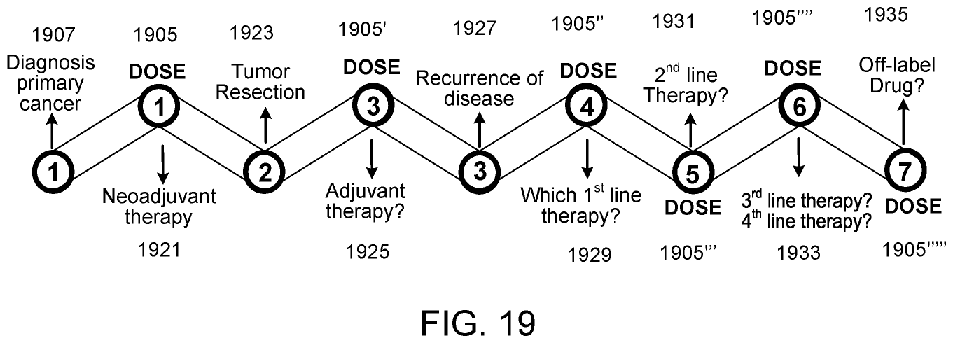

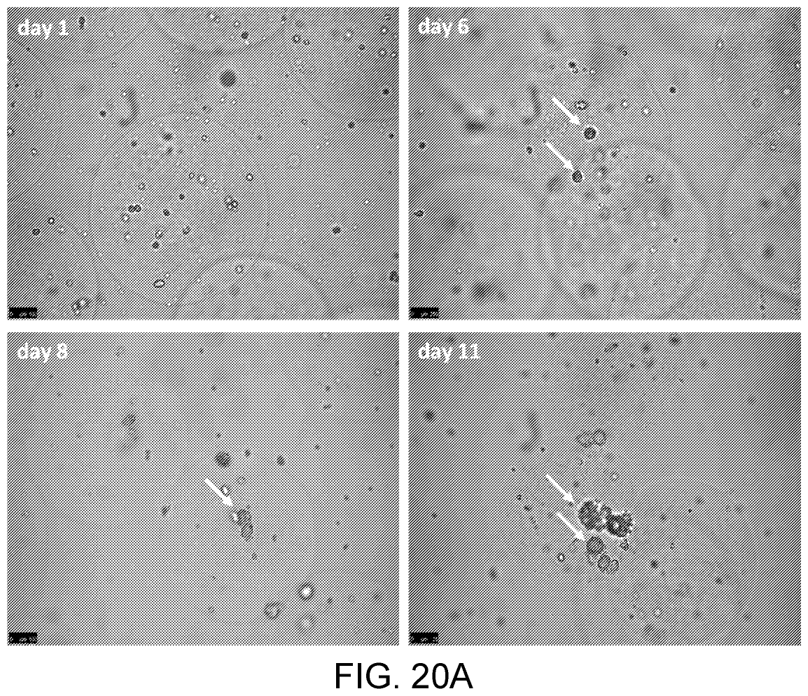

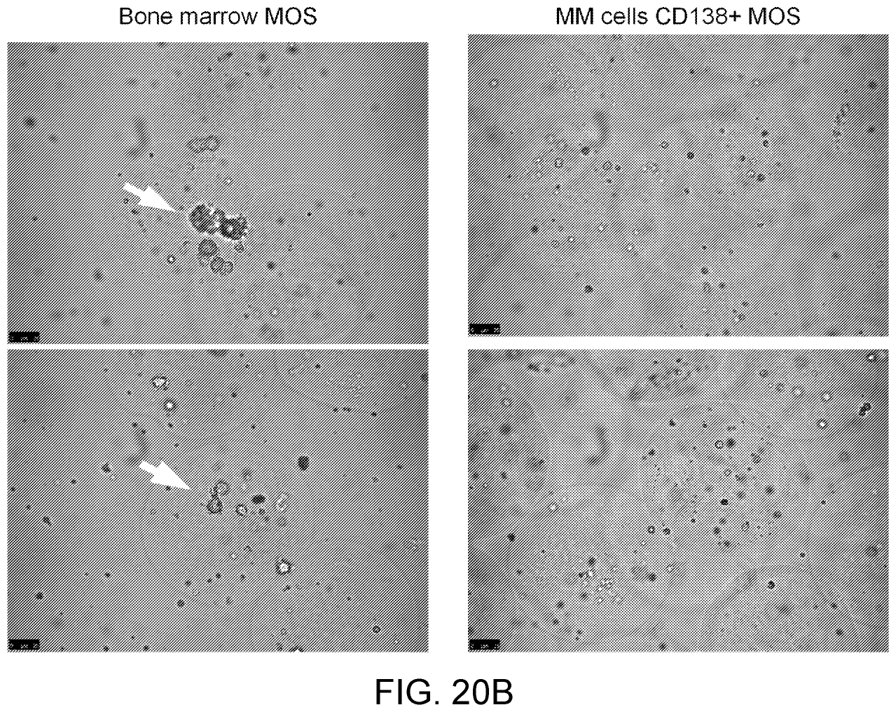

FIGS. 18 and 19 provide illustrations of therapeutic methods that use the methods and apparatuses, including the MOS, described herein. For precision and personalized medicine, these methods and apparatuses can be used as a clinical indicator for appropriate drug selection to improve clinical outcome and drug response. In some embodiments, a patient diagnosed with cancer (e.g., MM) can have a biopsy taken for histopathology and for screening of a plurality of MOS formed from the biopsy using a method as described herein. Within about 7 to 10 days, the screening can be performed from the biopsy to identify the most effective standard-of-care therapy so the patient can start treatment within about 14 days.

An example of this is illustrated in FIG. 18. In this example, a tumor can be identified at day 0 (e.g., by CT scan) 1801, a biopsy can be taken 1803 at day 5, and on the same day hundreds, thousands, or tens of thousands of MOS can be generated. The MOS can be cultured for about 1 to 5 days and then screened 1805 to identify one or more drug compositions that can be used. This same step (forming the MOS and screening) can be used to guide precision medicine at multiple clinical decision points throughout disease progression. In this example, therapy using the identified one or more drug compositions can be started 1809 on day 14, and the patient can later be monitored during the course of treatment (e.g., with a follow-up CT scan on about day 90) to confirm that the tumor is responding to the treatment 1811. If so, the therapy can be continued 1813 and the ongoing progress monitored 1815.

The use of MOS in assays can be repeated at multiple points throughout the course of patient treatment. This is illustrated in FIG. 19. For example, when a patient is first diagnosed 1907 with a resectable primary tumor, this technique (e.g., generation of MOS and screening 1905) can be used to determine the most effective neoadjuvant therapy 1921. Thus, a biopsy can be taken and hundreds, thousands, or tens of thousands of MOS can be formed and screened with a panel of potential drug compositions. Once the primary tumor is resected 1923, this technique 1905′ can indicate whether and which adjuvant therapy should be chosen 1925. If recurrence or metastasis happens after the surgical removal of the primary tumor 1927, the same technique (e.g., generating and screening MOS from a fresh biopsy 1905″, 1905′″, 1905″″) can be used to guide standard-of-care therapy, including first 1929, second 1931, and third line 1933 therapies. If the patient eventually becomes tolerant or resistant to all standard-of-care therapy, this technique 1905′″″ can be performed to identify off-label drugs to treat resistant tumors 1935. This technique can also be used as a companion diagnostic to identify patients for a specific treatment. Lastly, the technique can be used to derive and preserve patient-derived MOS to establish an organosphere-base living cancer bank for screening, genomic profiling, new drug discovery, drug testing, and/or clinical trial design.

Because the generation of a large number of MOS can be done relatively low-invasively (e.g., by resection or biopsy), and can be used to provide reasonably fast results from screening, the methods provided herein can be readily adapted for standard of care. For example, the volume of cellular material from the tissue (e.g., biopsy) input typically is quite small, and can be placed into a volume of, for example, about 10 μL to about 5 mL.

In general, the use of the MOS described herein for screening can be automated or manually performed. Virtually any screening technique can be used, including imaging by one or more of confocal microscopy, fluorescent microscopy, liquid lens, holography, sonar, bright and dark field imaging, laser, planar laser sheet, and high-throughput embodiments of image-based analysis methods (e.g., using computer vision and/or supervised or unsupervised models, such as CNN). Downstream screening can include sampling the culture media and/or performing genetic or protein screening (e.g., scRNA-seq, ATAC-seq, proteomics, etc.) on cells from the MOS.

### EXEMPLARY EMBODIMENTS

Embodiment 1 is a MicroOrganoSphere comprising bone marrow cells from a mammal with multiple myeloma (MM).

Embodiment 2 is the MicroOrganoSphere of embodiment 1, wherein the MicroOrganoSphere comprises about 50 to about 150 cells.

Embodiment 3 is the MicroOrganoSphere of embodiment 1, wherein the MicroOrganoSphere comprises about 75 to about 125 cells.

Embodiment 4 is the MicroOrganoSphere of embodiment 1, wherein the MicroOrganoSphere comprises about 100 cells.

Embodiment 5 is the MicroOrganoSphere of any one of embodiments 1 to 4, wherein the cells comprise cancer cells, stromal cells, stem cells, immune cells, or any combination thereof.

Embodiment 6 is the MicroOrganoSphere of embodiment 5, wherein the MicroOrganoSphere comprises cancer cells and stromal cells at a ratio of less than about 1:4.

Embodiment 7 is the MicroOrganoSphere of embodiment 5, wherein the immune cells comprise at least one macrophage.

Embodiment 8 is the MicroOrganoSphere of any one of embodiments 1 to 6, wherein the MicroOrganoSphere comprises a solubilized basement membrane matrix.

Embodiment 9 is a composition comprising the MicroOrganoSphere of any one of embodiments 1 to 8 in a culture medium.

Embodiment 10 is the composition of embodiment 9, wherein the culture medium comprises a solubilized basement membrane matrix.

Embodiment 11 is the composition of embodiment 10, comprising about 1% of the solubilized basement membrane matrix.

Embodiment 12 is the composition of any one of embodiments 9 to 11, further comprising an immiscible fluid.

Embodiment 13 is the MicroOrganoSphere of embodiment 12, wherein the immiscible fluid is an oil.

Embodiment 14 is a method for making MicroOrganoSpheres (MOS), where the method comprises:
receiving a bone marrow sample from a mammal having MM;
refining the bone marrow sample to form a refined sample; and
forming a population of MOS from the refined sample by:
   driving an unpolymerized fluid mixture through one or more channels of a microfluidics apparatus, wherein the unpolymerized fluid mixture comprises the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow,
   forming a plurality of droplets comprising the unpolymerized fluid mixture within the microfluidics apparatus, and
   polymerizing the fluid matrix material to form the MOS, wherein the MOS each have a diameter of between 50 and 500 μm with between 30 and 150 cells distributed therein.

Embodiment 15 is the method of embodiment 14, further comprising driving an immiscible fluid through another channel of the microfluidics apparatus, such that the immiscible fluid is combined with the unpolymerized fluid mixture prior to forming the plurality of droplets, wherein the droplets comprise the unpolymerized fluid mixture and the immiscible fluid.

Embodiment 16 is the method of embodiment 15, wherein the immiscible fluid is an oil.

Embodiment 17 is the method of any one of embodiments 14 to 16, wherein forming the population of MOS further comprises sorting the MOS based on cell number and/or droplet size.

Embodiment 18 is the method of embodiment 17, wherein the sorting comprises optical sorting based on cell number and/or droplet size.

Embodiment 19 is the method of any one of embodiments 14 to 18, wherein forming the population of MOS comprises forming about 100 to about 600 MOS.

Embodiment 20 is the method of any one of embodiments 14 to 18, wherein forming the population of MSO comprises forming about 600 to about 1,000 MOS.

Embodiment 21 is the method of any one of embodiments 14 to 18, wherein forming the population of MOS comprises forming more than about 1,000 MOS.

Embodiment 22 is the method of any one of embodiments 14 to 21, wherein the microfluidics apparatus maintains a viscosity of the unpolymerized fluid mixture prior to forming the plurality of droplets.

Embodiment 23 is the method of any one of embodiments 14 to 22, wherein the microfluidics apparatus is configured to prevent clogging of the unpolymerized fluid mixture within the one or more channels.

Embodiment 24 is the method of embodiment 23, wherein the microfluidics apparatus is configured to prevent clogging by having channel diameters of 100 μm or greater.

Embodiment 25 is the method of any one of embodiments 14 to 24, wherein the microfluidics apparatus is configured maintain an approximately constant pressure within the one or more channels.

Embodiment 26 is the method of any one of embodiments 14 to 25, wherein the microfluidics apparatus maintains a constant flow rate within the one or more channels.

Embodiment 27 is the method of any one of embodiments 14 to 26, wherein a total length of a path taken by the unpolymerized fluid mixture before the forming of the plurality of droplets within the microfluidics apparatus is less than 10 cm.

Embodiment 28 is the method of any one of embodiments 14 to 27, wherein the MOS of the population of MOS have less than a 25% variation in size.

Embodiment 29 is the method of any one of embodiments 14 to 28, wherein the polymerizing comprises crosslinking the fluid matrix material.

Embodiment 30 is the method of any one of embodiments 14 to 29, wherein the fluid matrix material is chemically crosslinkable or photo-crosslinkable.

Embodiment 31 is the method of any one of embodiments 14 to 30, wherein the bone marrow sample comprises freshly biopsied cells.

Embodiment 32 is the method of embodiment 31, wherein the bone marrow sample was taken from the mammal within 24 hours of forming the MOS.

Embodiment 33 is the method of any one of embodiments 14 to 32, wherein the bone marrow sample comprises MM plasma cells, immune cells, stem cells, stromal cells, or any combination thereof.

Embodiment 34 is the method of embodiment 33, wherein the immune cells comprise one or more of T cells, B cells, macrophages, dendritic cells, NK cells, monocytic cells, and combinations thereof.

Embodiment 35 is the method of any one of embodiments 14 to 34, comprising flowing the unpolymerized fluid mixture through the one or more channels at a flow rate of about 0.01 milliliter (mL) per minute (min) to about 100 mL/min.

Embodiment 36 is a method of precision drug screening for personalized cancer therapy for MM, where the method comprises:
receiving a bone marrow sample from a mammal having MM;
refining the sample to form a refined sample;
forming a population of MOS from the refined sample by:
   driving an unpolymerized fluid mixture through one or more channels of a microfluidics apparatus, wherein the unpolymerized fluid mixture comprises the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow,
   forming a plurality of droplets comprising the unpolymerized fluid mixture within the microfluidics apparatus, and
   polymerizing the fluid matrix material to form the MOS, wherein the MOS each have a diameter of between 50 and 500 μm with between 1 and 500 cells distributed therein;
culturing the population of MOS for between 1-14 days; and
assaying one or more drug therapies using the population of MOS.

Embodiment 37 is the method of embodiment 36, further comprising driving an immiscible fluid through another channel of the microfluidics apparatus, such that the immiscible fluid is combined with the unpolymerized fluid mixture prior to forming the plurality of droplets, wherein the droplets comprise the unpolymerized fluid mixture and the immiscible fluid.

Embodiment 38 is the method of embodiment 37, wherein the immiscible fluid is an oil.

Embodiment 39 is the method of any one of embodiments 36 to 38, wherein the assaying comprises assaying, in parallel, a plurality of drug therapies by exposing one or more of the MOS to each drug therapy.

Embodiment 40 is the method of embodiment 39, comprising characterizing a response of the MOS to each of the plurality of drug therapies based on a response of the MOS to exposure to the plurality of drug therapies.

Embodiment 41 is the method of any one of embodiments 36 to 40, wherein a time between receiving the bone marrow sample and characterizing the response is less than 21 days.

Embodiment 42 is the method of any one of embodiments 36 to 41, wherein forming the population of MOS further comprises sorting the MOS based on cell number and/or droplet size.

Embodiment 43 is the method of any one of embodiments 36 to 42, wherein the sorting comprises optically sorting the MOS or based on cell number and/or droplet size.

Embodiment 44 is the method of any one of embodiments 36 to 43, wherein the assaying comprises assaying more than 10 different drug therapies.

Embodiment 45 is the method of any one of embodiments 36 to 44, wherein the one or more drug therapies include different concentrations of one or more drug, different combinations of two or more drugs, different ratios of two or more drugs, different carriers for one or more drug, and/or different dose times for one or more drugs.

Embodiment 46 is the method of any one of embodiments 36 to 45, wherein forming the population of MOS comprises forming about 100 to about 600 MOS.

Embodiment 47 is the method of any one of embodiments 36 to 45, wherein formation the population of MOS comprises forming about 600 to about 1,000 MOS.

Embodiment 48 is the method of any one of embodiments 36 to 45, wherein forming the population of MOS comprises forming more than 1,000 MOS.

Embodiment 49 is the method of any one of embodiments 36 to 48, wherein the microfluidics apparatus maintains a viscosity of the unpolymerized fluid mixture prior to forming the plurality of droplets.

Embodiment 50 is the method of any one of embodiments 36 to 49, wherein the microfluidics apparatus is configured to prevent clogging of the unpolymerized fluid mixture within the one or more channels.

Embodiment 51 is the method of embodiment 50, wherein the microfluidics apparatus is configured to prevent clogging by having channel diameters of 100 μm or greater.

Embodiment 52 is the method of any one of embodiments 36 to 51, wherein the microfluidics apparatus is configured maintain an approximately constant pressure within the one or more channels.

Embodiment 53 is the method of any one of embodiments 36 to 52, wherein the microfluidics apparatus maintains a constant flow rate within the one or more channels.

Embodiment 54 is the method of any one of embodiments 36 to 53, wherein a total length of a path taken by the unpolymerized fluid mixture before the forming of the plurality of droplets within the microfluidics apparatus is less than 10 cm.

Embodiment 55 is the method of any one of embodiments 36 to 54, further comprising measuring an effect of the one or more drug therapies on cells within the MOS.

Embodiment 56 is the method of any one of embodiments 36 to 55, further comprising determining that the mammal is still responding to a drug therapy of the one or more drug therapies after one or more administrations of the drug therapy, by receiving a second bone marrow sample after the mammal has been treated with the drug therapy and forming a second population of MOS from the second bone marrow sample, exposing at least some of the second population of MOS to the drug therapy, and measuring an effect of the drug therapy on cells within the at least some of the second population of MOS.

Embodiment 57 is the method of any one of embodiments 36 to 56, further comprising treating the mammal with a drug therapy of the one or more drug therapies.

Embodiment 58 is the method of any one of embodiments 36 to 57, wherein the MOS of the population of MOS have less than a 25% variation in size.

Embodiment 59 is the method of any one of embodiments 36 to 58, wherein the polymerizing comprises crosslinking the fluid matrix material.

Embodiment 60 is the method of any one of embodiments 36 to 59, wherein the fluid matrix material is chemically crosslinkable or photo-crosslinkable.

Embodiment 61 is the method of any one of embodiments 36 to 60, wherein the bone marrow sample comprises freshly biopsied cells.

Embodiment 62 is the method of embodiment 61, wherein the bone marrow sample was taken from the mammal within 24 hours of forming the MOS.

Embodiment 63 is the method of any one of embodiments 36 to 62, wherein the bone marrow sample comprises cancer cells, immune cells, stem cells, stromal cells, or any combination thereof.

Embodiment 64 is the method of embodiment 63, wherein the immune cells comprise one or more of T cells, B cells, macrophages, dendritic cells, NK cells, monocytic cells, and combinations thereof.

Embodiment 65 is the method of any one of embodiments 36 to 64, comprising flowing the refined bone marrow sample and the unpolymerized fluid matrix through the one or more channels at a flow rate of about 0.01 mL/min to about 100 mL/min.

The invention will be further described in the following examples, which do not limit the scope of the invention described in the claims.

### EXAMPLES

### Example 1—Preparation of MM MOS

Cells were isolated from fresh bone marrow samples (1-3 mL each) from MM patient biopsies. Briefly, red blood cells were lysed in the bone marrow samples, and 70% MATRIGEL® was added to the bone marrow cell pellet. MM MOS were generated at density of 30 cells per MOS. The MOS culture medium [RPMI, 10% fetal bovine serum (FBS), 1% penicillin/streptomycin, 200 ng/ml granulocyte macrophage colony stimulating factor (GM-CSF), and 100 ng/ml interleukin-6 (IL-6)] was changed twice a week. Representative images showing MM MOS formation on days 1, 6, 8, and 11 are shown in FIG. 20A, with MM MOS indicated by the white arrows. Day 11 MM MOS were subjected to flow cytometry in order to determine what cell populations were present. CD138+ cells isolated from bone marrow cells using bead selection did not form MOS, however (FIG. 20B), indicating that the presence of stromal cells was required for MOS formation.

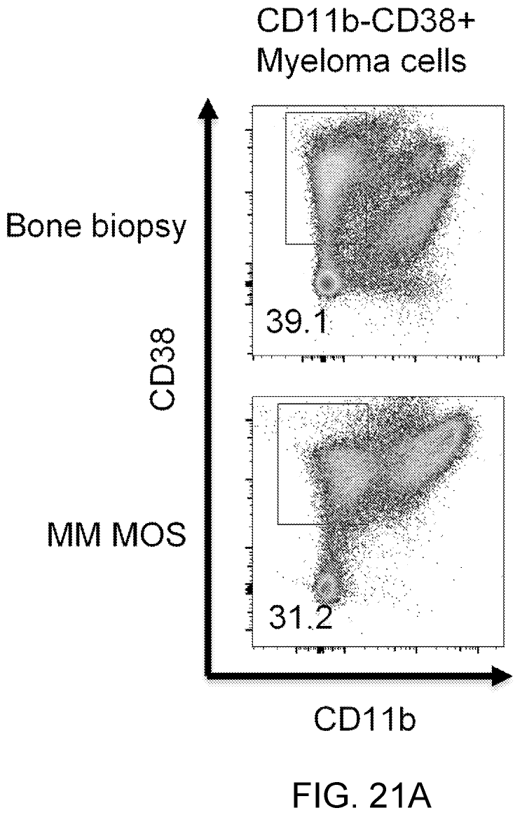

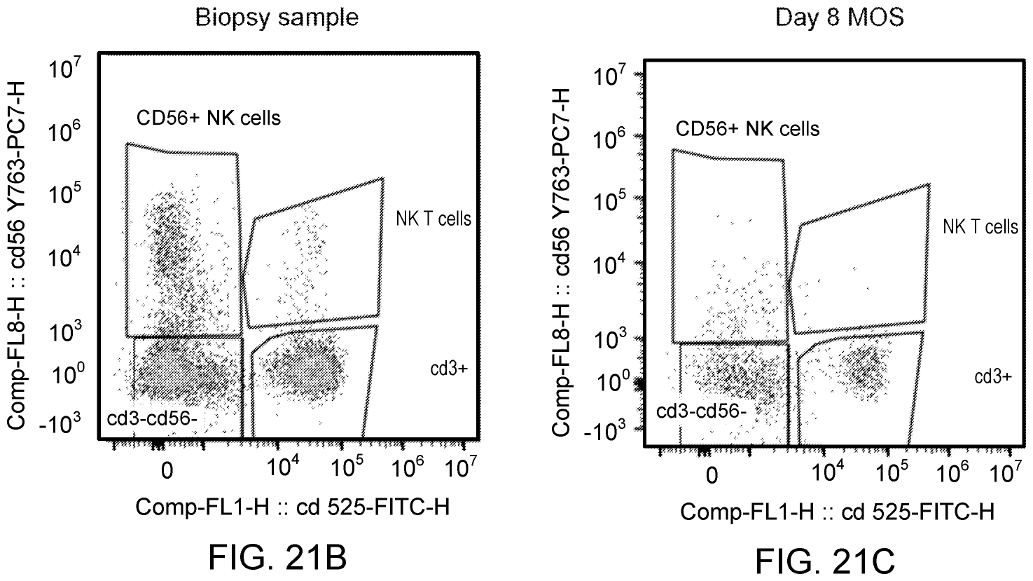

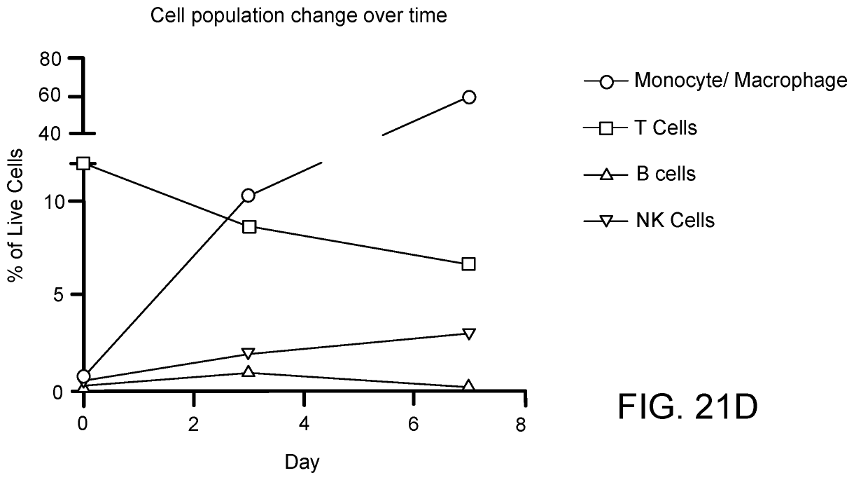

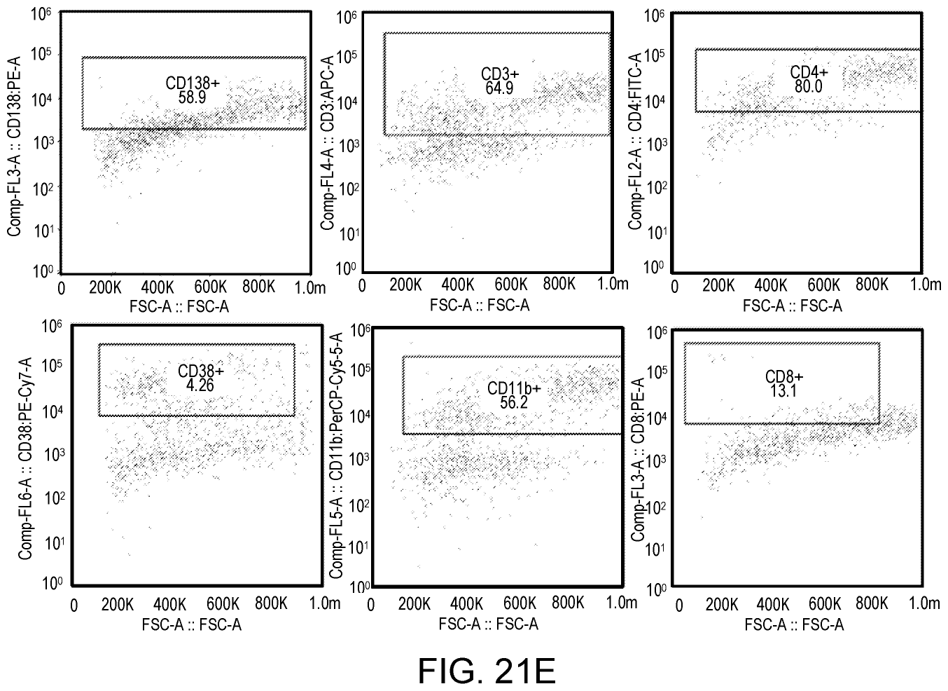

As shown in FIG. 21A, the major bone marrow cell populations were preserved in established MM MOS after 9 days in vitro. The myeloma cell population was demonstrated to be CD11b− and CD38+. Flow cytometry also showed that the major immune cell populations were preserved in day 8 MOS (FIG. 21C) as compared to bone marrow biopsy (FIG. 21B). Overall cell percentages over time during MOS culture are plotted in the graph shown in FIG. 21D. Additional studies demonstrated that populations of plasma cells and MM cells (CD138⁺ and CD38⁺, respectively), T cells (CD3⁺, CD4⁺, and CD8⁺, respectively), and dendritic cells (CD11b⁺) were all present in the MOS (FIG. 21E).

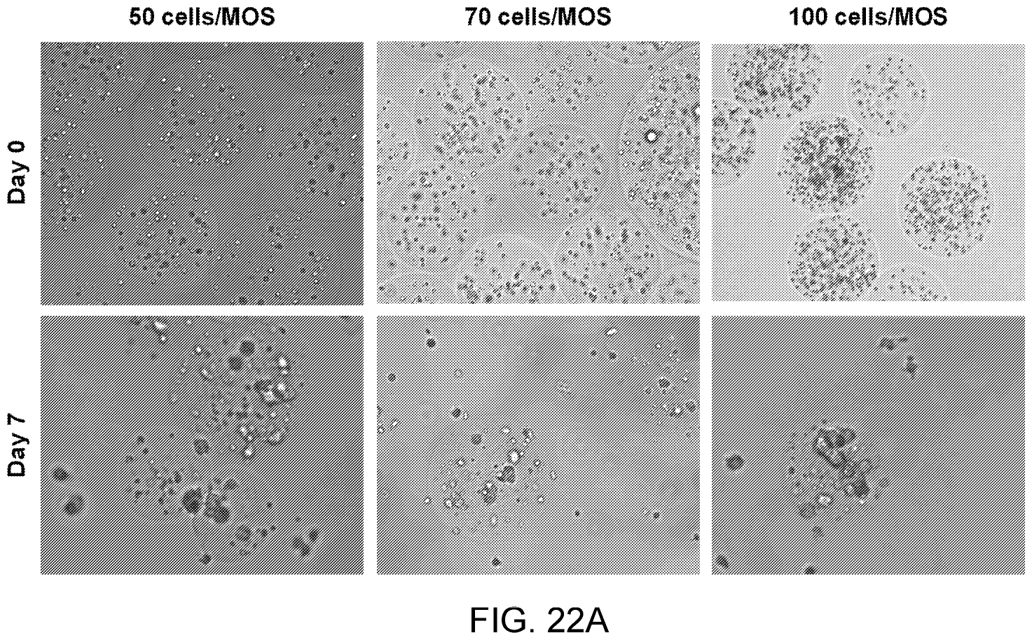

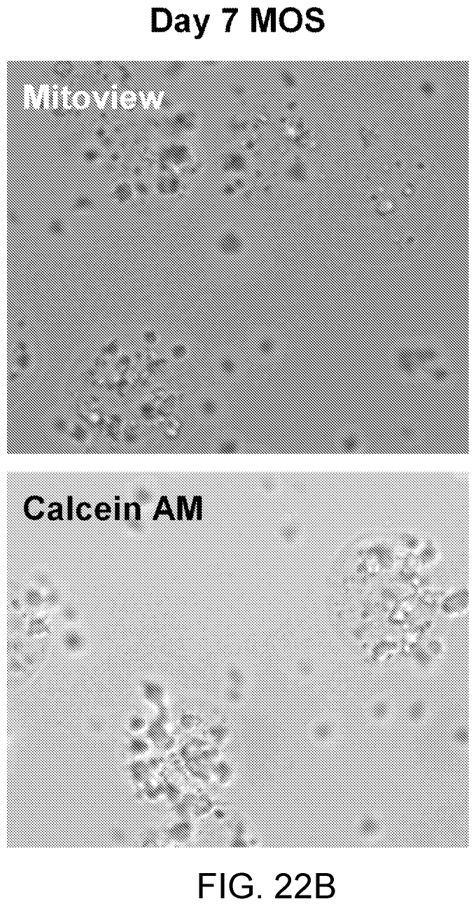

FIG. 22A includes representative images showing MOS containing 50, 70, or 100 cells, at days 0 and 7. FIG. 22B includes representative images of day 7 MOS stained for live cells.

MOS containing 50, 70, or 100 cells each also were prepared from a bone marrow biopsy sample containing about 8×10⁶ cells. Representative images showing these MOS are presented in FIG. 22A, and live-dead staining of representative day 7 MOS are shown in FIG. 22B, demonstrating about 95% viability.

### Example 2—Effect of Drug Treatment on MM MOS

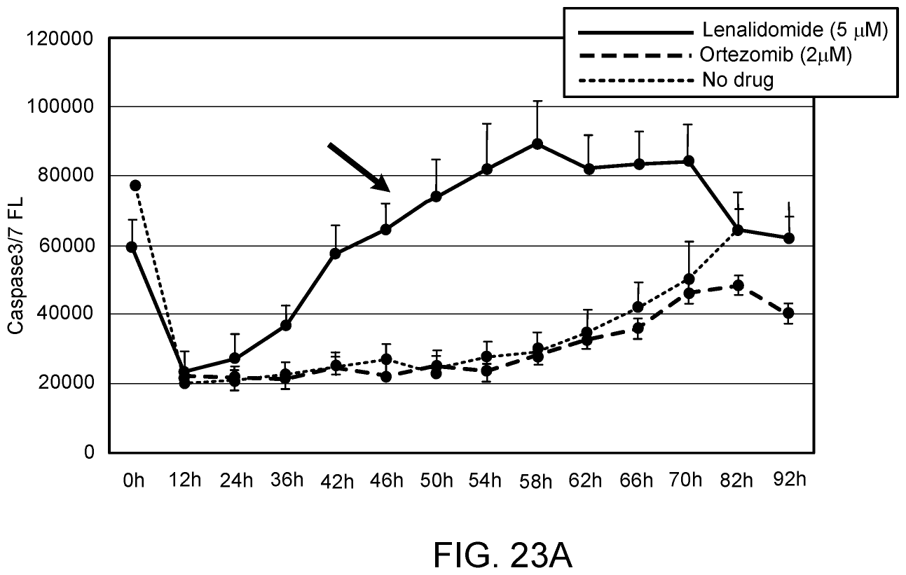

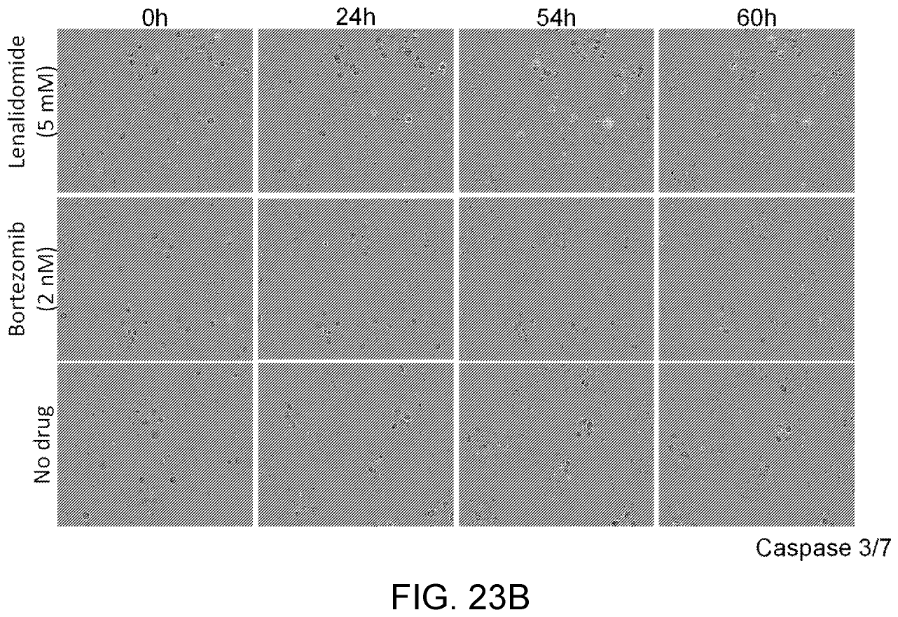

Studies were conducted to determine the effects of various MM drugs on the MM MOS. For example, day 11 MM MOS were treated with 5 μM lenalidomide or 2 nM bortezomib for 92 hours, with no medium change during the drug treatment. Caspase 3/7 green dye was included in the medium to monitor apoptosis, and fluorescence was measured. These studies demonstrated that MM MOS death was increased with the lenalidomide treatment, but not with the negative control or the bortezomib treatment (FIG. 23A). In addition, INCUCYTE® images taken every 2 hours over the course of treatment revealed that the MM MOS responded to lenalidomide as early as 24 hours after the onset of treatment (FIG. 23B).

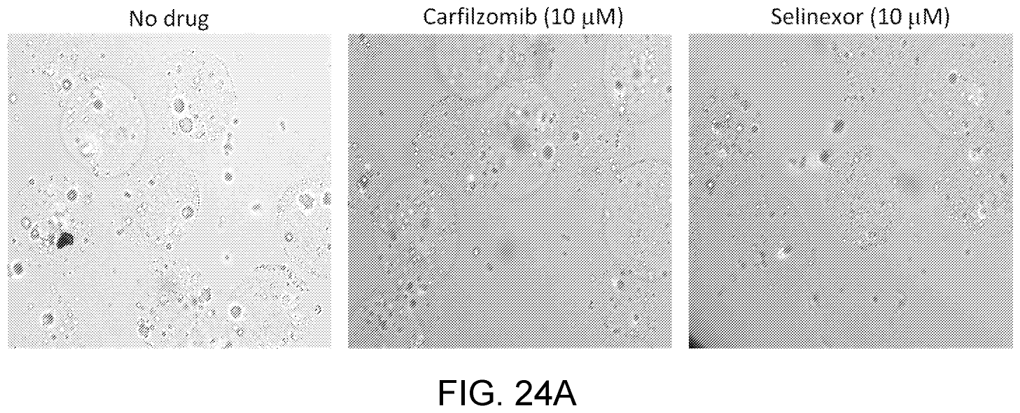

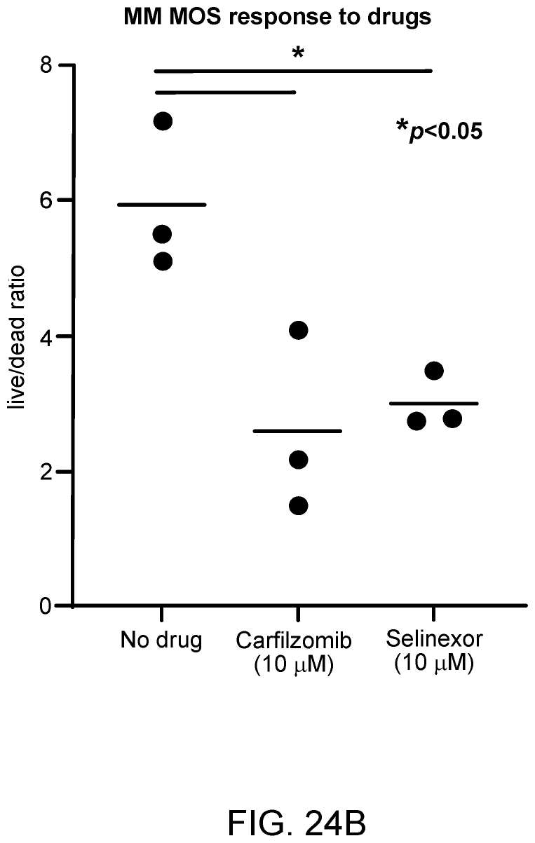

Further studies were conducted using 10 μM carfilzomib or 10 μM selinexor treatment beginning on day 9 of MOS generation from MM patient bone marrow biopsy samples. After 4 days of treatment, live/dead dyes (calcein AM—green and ethidium homodimer—red) were added to the wells for a 30-minute incubation. Images were taken (FIG. 24A) using an EVOS™ M7000 Imaging System (ThermoFisher Scientific; Waltham, MA). A Xilis AI algorithm was used to calculate each individual organoid's fluorescence signal on both channels. Live/dead ratios were calculated, and dot plot data are presented in FIG. 24B as the average ratio from each well. A significantly reduced live/dead ratio was observed in the MM MOS treated with either carfilzomib or selinexor, as compared to the negative control.

### Example 3—MM MOS Can Survive Freezing and Thawing

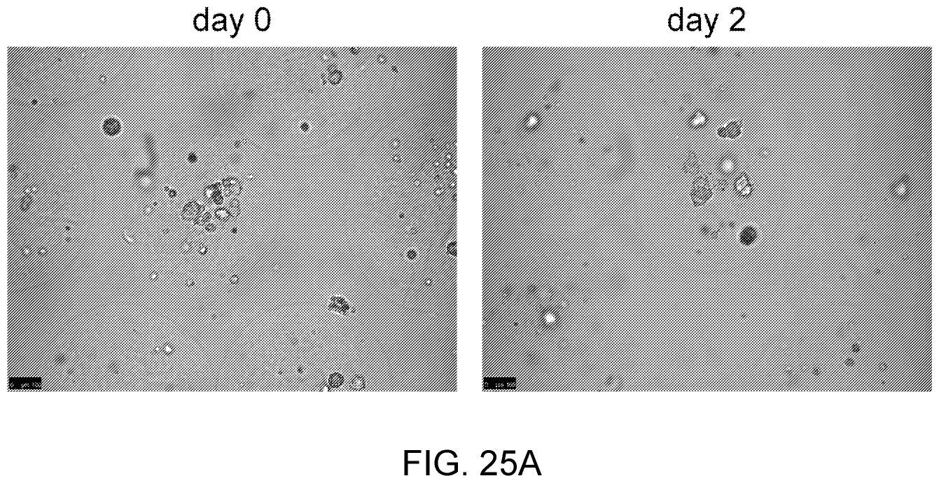

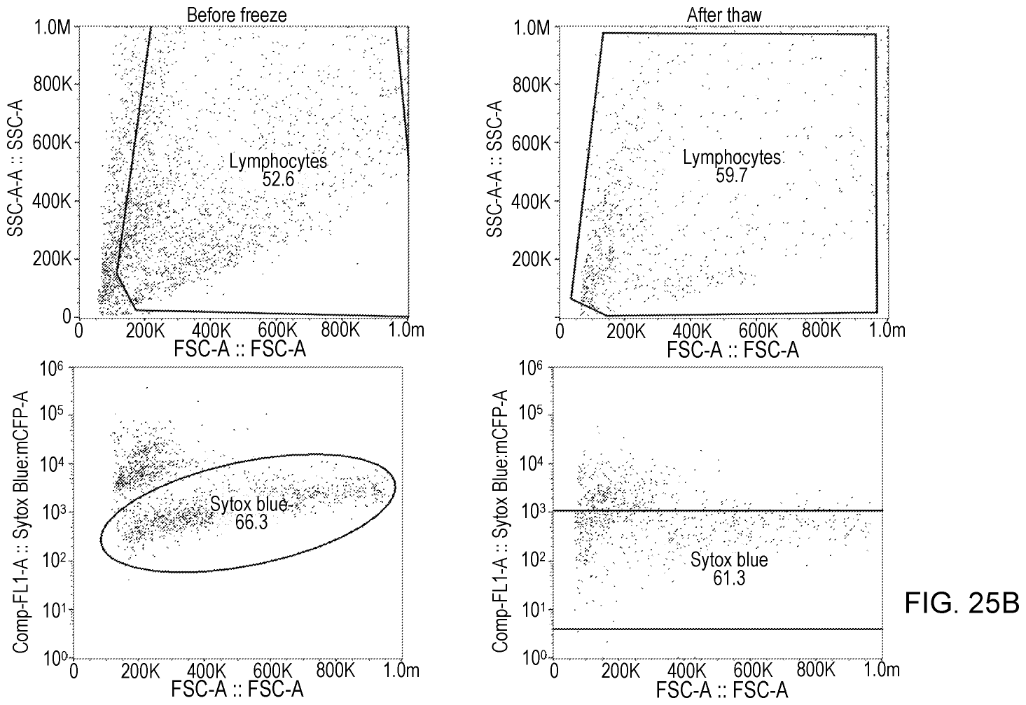

MM MOS were frozen without breaking the MATRIGEL® structure. After two days in liquid nitrogen, the frozen MM MOS were thawed in culture medium in a 37° C. water bath. The droplet structure was maintained after thawing (FIG. 25A), and viability was confirmed by SYTOX™ blue-measurement via flow cytometry (FIG. 25B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A spherical droplet comprising bone marrow cells from a mammal with multiple myeloma (MM), wherein said spherical droplet has a diameter of between 50 μm and 500 μm.

2. The spherical droplet of claim 1, wherein the MicroOrganoSphere comprises about 50 to about 150 cells.

3-4. (canceled)

5. The spherical droplet of claim 1, wherein the cells comprise cancer cells, stromal cells, stem cells, immune cells, or any combination thereof.

6-7. (canceled)

8. The spherical droplet of claim 1, wherein the spherical droplet comprises a solubilized basement membrane matrix.

9. A composition comprising the spherical droplet of claim 1 in a culture medium.

10. The composition of claim 9, wherein the culture medium comprises a solubilized basement membrane matrix.

11. (canceled)

12. The composition of claim 9, further comprising an immiscible fluid.

13. (canceled)

14. A method for making spherical droplets comprising bone marrow cells from a mammal with multiple myeloma (MM), the method comprising:
receiving a bone marrow sample from a mammal having MM;
refining the bone marrow sample to form a refined sample; and
forming a population of spherical droplets from the refined sample by:
driving an unpolymerized fluid mixture through one or more channels of a microfluidics apparatus, wherein the unpolymerized fluid mixture comprises the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow,
forming a plurality of droplets comprising the unpolymerized fluid mixture within the microfluidics apparatus, and
polymerizing the fluid matrix material to form the spherical droplets, wherein the spherical droplets each have a diameter of between 50 and 500 μm with between 30 and 150 cells distributed therein.

15. The method of claim 14, further comprising driving an immiscible fluid through another channel of the microfluidics apparatus, such that the immiscible fluid is combined with the unpolymerized fluid mixture prior to forming the plurality of droplets, wherein the droplets comprise the unpolymerized fluid mixture and the immiscible fluid.

16. (canceled)

17. The method of claim 14, wherein forming the population of spherical droplets further comprises sorting the spherical droplets based on cell number and/or droplet size.

18-20. (canceled)

21. The method of claim 14, wherein forming the population of spherical droplets comprises forming more than about 1,000 spherical droplets.

22. The method of claim 14, wherein the microfluidics apparatus maintains a viscosity of the unpolymerized fluid mixture prior to forming the plurality of droplets, and/or
wherein the microfluidics apparatus is configured to prevent clogging of the unpolymerized fluid mixture within the one or more channels, and/or
wherein the microfluidics apparatus is configured to maintain an approximately constant pressure within the one or more channels, and/or
wherein the microfluidics apparatus maintains a constant flow rate within the one or more channels, and/or
wherein a total length of a path taken by the unpolymerized fluid mixture before the forming of the plurality of droplets within the microfluidics apparatus is less than 10 cm.

23-28. (canceled)

29. The method of claim 14, wherein the polymerizing comprises crosslinking the fluid matrix material.

30. (canceled)

31. The method of claim 14, wherein the bone marrow sample comprises freshly biopsied cells taken from the mammal within 24 hours of forming the spherical droplets.

32. (canceled)

33. The method of claim 14, wherein the bone marrow sample comprises MM plasma cells, immune cells, stem cells, stromal cells, or any combination thereof.

34-35. (canceled)

36. A method of precision drug screening for personalized cancer therapy for MM, the method comprising:
receiving a bone marrow sample from a mammal having MM;
refining the sample to form a refined sample;
forming a population of spherical droplets from the refined sample by:
driving an unpolymerized fluid mixture through one or more channels of a microfluidics apparatus, wherein the unpolymerized fluid mixture comprises the refined sample and an unpolymerized fluid matrix material, wherein the microfluidics apparatus controls a pressure, flow rate, or pressure and flow rate within the one or more channels so that the refined sample and the unpolymerized fluid matrix material travels through the one or more channels in laminar flow,
forming a plurality of droplets comprising the unpolymerized fluid mixture within the microfluidics apparatus, and
polymerizing the fluid matrix material to form the spherical droplets, wherein the spherical droplets each have a diameter of between 50 and 500 μm with between 1 and 500 cells distributed therein;
culturing the population of spherical droplets for between 1-14 days; and
assaying one or more drug therapies using the population of spherical droplets.

37. The method of claim 36, further comprising driving an immiscible fluid through another channel of the microfluidics apparatus, such that the immiscible fluid is combined with the unpolymerized fluid mixture prior to forming the plurality of droplets, wherein the droplets comprise the unpolymerized fluid mixture and the immiscible fluid.

38. (canceled)

39. The method of claim 36, wherein the assaying comprises assaying, in parallel, a plurality of drug therapies by exposing one or more of the spherical droplets to each drug therapy, and characterizing a response of the spherical droplets to each of the plurality of drug therapies based on a response of the spherical droplets to exposure to the plurality of drug therapies.

40. (canceled)

41. The method of claim 36, wherein a time between receiving the bone marrow sample and characterizing the response is less than 21 days.

42-44. (canceled)

45. The method of claim 36, wherein the one or more drug therapies include different concentrations of one or more drug, different combinations of two or more drugs, different ratios of two or more drugs, different carriers for one or more drug, and/or different dose times for one or more drugs.

46-47. (canceled)

48. The method of claim 36, wherein forming the population of spherical droplets comprises forming more than 1,000 spherical droplets.

49. The method of claim 36, wherein the microfluidics apparatus maintains a viscosity of the unpolymerized fluid mixture prior to forming the plurality of droplets and/or
wherein the microfluidics apparatus is configured to prevent clogging of the unpolymerized fluid mixture within the one or more channels; and/or
wherein the microfluidics apparatus is configured to maintain an approximately constant pressure within the one or more channels; and/or
wherein the microfluidics apparatus maintains a constant flow rate within the one or more channels; and/or
wherein a total length of a path taken by the unpolymerized fluid mixture before the forming of the plurality of droplets within the microfluidics apparatus is less than 10 cm.

50-65. (canceled)
